(19) United States
(12) Patent Application Publication
(10) Pub. No.: US 2023/0200716 A1
(43) Pub. Date: Jun. 29, 2023

(54) USE OF NEUROMELANIN-SENSITIVE MRI AS A BIOMARKER OF DOPAMINE FUNCTION

(71) Applicants: The Trustees of Columbia University in the City of New York, New York, NY (US); THE RESEARCH FOUNDATION FOR MENTAL HYGIENE, INC., Menands, NY (US)

(72) Inventors: Guillermo Horga HERNANDEZ, New York, NY (US); Clifford Mills CASSIDY, Ottawa (CA); Kenneth WENGLER, New York, NY (US)

(21) Appl. No.: 18/171,276

(22) Filed: Feb. 17, 2023

Related U.S. Application Data
(63) Continuation of application No. PCT/US2021/046231, filed on Aug. 17, 2021.
(60) Provisional application No. 63/066,744, filed on Aug. 17, 2020.

(51) Int. Cl. A61B 5/00; A61B 5/055; A61B 5/145; A61B 5/16; G01R 33/483

(52) U.S. Cl. CPC A61B 5/4064; A61B 5/055; A61B 5/14546; A61B 5/16; G01R 33/483; A61B 2576/026

(57) ABSTRACT

The subject matter disclosed herein relates to a method for determining dopamine function in a subject, the method comprising acquiring one or more neuromelanin-Magnetic Resonance Imaging (NM-MRI) scans of the subject's dopamine-associated brain region of interest.

### GOVERNMENT SUPPORT

The work described herein was supported in whole, or in part, by National Institutes Health Grant Nos. R01MH114965, R01MH117323, R01DA020855, and UL1TR001873.Thus, the United States Government has certain rights to the invention.

### RELATED APPLICATIONS

The present application claims priority to, and the benefit of, U.S. Provisional Patent Application No. 63/066,744, filed Aug. 17, 2020, the content of which is incorporated by reference in its entirety.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Magnetic resonance imaging (MRI) is an imaging technique used in medicine to form pictures of the anatomy and the physiological processes of the brain. MRI scanners use strong magnetic fields, magnetic field gradients, and radio waves to generate images.

### SUMMARY OF THE INVENTION

In certain aspects, the invention provides a method for determining dopamine function in a subject, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; determining a NM concentration in the brain region of interest using voxelwise analysis based on the imaging information; and determining the dopamine function based on the NM concentration; wherein the determining of the dopamine function comprises: (1) if the one or more NM-MRI scans has increased NM signal compared to a one or more control scans then dopamine function is increased; or (2) if the one or more NM-MRI scans has decreased NM signal compared to a one or more control scans then dopamine function is decreased.

In some embodiments, the voxelwise analysis comprises determining at least one topographical pattern within the brain region of interest. In some embodiments, the at least one topographical pattern includes at least one pattern comprising a change in cell number in the brain region of interest.

In some embodiments, the one or more acquired NM-MRI scans are related to the subject's performance on a cognitive task. In some embodiments, the cognitive task assesses catecholamine-related processes. In some embodiments, the catecholamine-related processes comprise dopamine- related processes. In some embodiments, the catecholamine-related processes comprise reward processing.

In some embodiments, the brain region is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In some embodiments, the subject has or is suspected of having one or more dopamine function-related disorder. the subject has or is suspected of having schizophrenia spectrum disorders. In some embodiments, the subject has or is suspected of having psychotic illness. In some embodiments, the subject has or is suspected of having addiction disorder. In some embodiments, the subject has or is suspected of having depression. In some embodiments, the subject has or is suspected of having late-life depression. In some embodiments, the subject has or is suspected of having bipolar disorder.

In some embodiments, the subject has or is suspected of having Huntington's disease. In some embodiments, the subject has or is suspected of having Parkinson's disease. In some embodiments, the subject has or is suspected of having one or more movement disorders. In some embodiments, the subject has or is suspected of having psychomotor slowing. In some embodiments, the subject has or is suspected of having one or more neuropsychiatric disorders. In some embodiments, the subject has or is suspected of having a cocaine use disorder.

In certain aspects, the invention provides a method for determining if a subject has or is at risk of developing a neuropsychiatric disorder, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; and determining a NM concentration in the brain region of interest using voxelwise analysis based on the imaging information; wherein the determining if a subject has or is at risk of developing a neuropsychiatric disorder comprises: (1) if the one or more NM-MRI scans has an altered NM signal compared to a one or more control scans without a neuropsychiatric disorder then the subject has or is at risk of developing a neuropsychiatric disorder; or (2) if the one or more NM-MRI scans has a NM signal comparable to the signal of a one or more control scans without a neuropsychiatric disorder then the subject does not have or is not at risk of developing a neuropsychiatric disorder.

In some embodiments, the voxelwise analysis for determining if a subject has or is at risk of developing a neuropsychiatric disorder comprises determining at least one topographical pattern within the brain region of interest. In some embodiments, the at least one topographical pattern includes at least one pattern comprising a change in cell number in the brain region of interest.

In some embodiments, the one or more acquired NM-MRI scans for determining if a subject has or is at risk of developing a neuropsychiatric disorder are related to the subject's performance on a cognitive task. In some embodiments, the cognitive task assesses catecholamine-related processes. In some embodiments, the catecholamine-related processes comprise dopamine-related processes. In some embodiments, the catecholamine-related processes comprise reward processing. In some embodiments, the one or more NM-MRI scans has increased signal compared to a one or more control scans without a neuropsychiatric disorder. In some embodiments, the one or more NM-MRI scans has decreased signal compared to a one or more control scans without a neuropsychiatric disorder.

In some embodiments, the brain region for determining if a subject has or is at risk of developing a neuropsychiatric disorder is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In some embodiments, the neuropsychiatric disorder comprises schizophrenia spectrum disorders. In some embodiments, the neuropsychiatric disorder comprises psychotic illness. In some embodiments, the neuropsychiatric disorder comprises addiction. In some embodiments, the neuropsychiatric disorder comprises depression. In some embodiments, the neuropsychiatric disorder comprises late-life depression. In some embodiments, the neuropsychiatric disorder comprises bipolar disorder.

In some embodiments, the neuropsychiatric disorder comprises Huntington's disease. In some embodiments, the neuropsychiatric disorder comprises psychomotor slowing. In some embodiments, the neuropsychiatric disorder comprises Parkinson's disease. In some embodiments, the neuropsychiatric disorder comprises one or more movement disorders. In some embodiments, the neuropsychiatric disorder comprises cocaine use disorder.

In certain aspects, the invention provides a method for determining if a subject has or is at risk of developing a cognitive disorder, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; and determining a NM concentration in the brain region of interest using voxelwise analysis based on the imaging information; wherein the determining if a subject has or is at risk of developing a cognitive disorder comprises: (1) if the one or more NM-MRI scans has altered signal compared to a one or more control scans without a cognitive disorder then the subject has or is at risk of developing a cognitive disorder; or (2) if the one or more NM-MRI scans has signal comparable to the signal of a one or more control scans without a cognitive disorder then the subject does not have or is not at risk of developing a cognitive disorder.

In some embodiments, the voxelwise analysis for determining if a subject has or is at risk of developing a cognitive disorder comprises determining at least one topographical pattern within the brain region of interest. In some embodiments, the at least one topographical pattern includes at least one pattern comprising a change in cell number in the brain region of interest.

In some embodiments, the one or more acquired NM-MRI scans for determining if a subject has or is at risk of developing a cognitive disorder are related to the subject's performance on a cognitive task. In some embodiments, the cognitive task assesses catecholamine-related processes. In some embodiments, the catecholamine-related processes comprise dopamine-related processes. In some embodiments, the catecholamine-related processes comprise reward processing.

In some embodiments, the one or more NM-MRI scans for determining if a subject has or is at risk of developing a cognitive disorder has increased signal compared to a one or more control scans without a neuropsychiatric disorder. In some embodiments, the one or more NM-MRI scans has decreased signal compared to a one or more control scans without a neuropsychiatric disorder.

In some embodiments, the brain region for determining if a subject has or is at risk of developing a cognitive disorder is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In some embodiments, the cognitive disorder comprises a neurocognitive disorder. In some embodiments, the cognitive disorder comprises memory dysfunction.

In certain aspects, the invention provides a method for determining if a subject has or is at risk of developing an addiction disorder, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; and determining a NM concentration in the brain region of interest using voxelwise analysis based on the imaging information; wherein the determining if a subject has or is at risk of developing an addiction disorder comprises: (1) if the one or more NM-MRI scans has altered NM signal compared to a one or more control scans without an addiction disorder then the subject has or is at risk of developing an addiction disorder; or (2) if the one or more NM-MRI scans has a NM signal comparable to a one or more control scans without addiction disorder then the subject does not have or is not at risk of developing an addiction disorder.

In some embodiments, the voxelwise analysis for determining if a subject has or is at risk of developing an addiction disorder comprises determining at least one topographical pattern within the brain region of interest. In some embodiments, the at least one topographical pattern includes at least one pattern comprising a change in cell number in the brain region of interest.

In some embodiments, the one or more acquired NM-MRI scans for determining if a subject has or is at risk of developing an addiction disorder are related to the subject's performance on a cognitive task. In some embodiments, the cognitive task assesses catecholamine-related processes. In some embodiments, the catecholamine-related processes comprise dopamine-related processes. In some embodiments, the catecholamine-related processes comprise reward processing. In some embodiments, the one or more NM-MRI scans has increased signal compared to a one or more control scans without a neuropsychiatric disorder. In some embodiments, the one or more NM-MRI scans has decreased signal compared to a one or more control scans without a neuropsychiatric disorder.

In some embodiments, the brain region is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In some embodiments, the addiction disorder comprises cocaine use disorder. In some embodiments, the addiction disorder comprises nicotine use disorder. In some embodiments, the addiction disorder comprises alcohol use disorder. In some embodiments, the addiction disorder comprises methamphetamine use disorder. In some embodiments, the addiction disorder comprises opiates use disorder. In some embodiments, the addiction disorder comprises behavioral addictions.

In certain aspects, the invention provides a method of determining if a subject has or is at risk of developing Parkinson's disease, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; and determining a NM concentration in the brain region of interest using voxelwise analysis based on the imaging information; wherein the determining if a subject has or is at risk of developing Parkinson's disease comprises: (1) if the one or more NM-MRI scans has a decreased NM signal compared to a one or more control scans without Parkinson's disease then the subject has or is at risk of developing Parkinson's disease; or (2) if the one or more NM-MRI scans has a NM signal comparable to the signal of a one or more control scans without Parkinson's disease then the subject does not have or is not at risk of developing Parkinson's disease.

In some embodiments, the voxelwise analysis for determining if a subject has or is at risk of developing Parkinson's disease comprises determining at least one topographical pattern within the brain region of interest. In some embodiments, the at least one topographical pattern includes at least one pattern comprising a change in cell number in the brain region of interest.

In some embodiments, the one or more acquired NM-MRI scans for determining if a subject has or is at risk of developing Parkinson's disease are related to the subject's performance on a cognitive task. In some embodiments, the cognitive task assesses catecholamine-related processes. In some embodiments, the catecholamine-related processes comprise dopamine-related processes. In some embodiments, the catecholamine-related processes comprise reward processing.

In some embodiments, the brain region is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In certain aspects, the invention provides a method of determining if a subject has or is at risk of developing psychomotor slowing, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; and determining a NM concentration in the brain region of interest using voxelwise analysis based on the imaging information; wherein the determining if a subject has or is at risk of developing psychomotor slowing comprises: (1) if the one or more NM-MRI scans has a decreased NM signal compared to a one or more control scans without psychomotor slowing then the subject has or is at risk of developing psychomotor slowing; or (2) if the one or more NM-MRI scans has a NM signal comparable to the signal of a one or more control scans without psychomotor slowing then the subject does not have or is not at risk of developing psychomotor slowing.

In some embodiments, the subject has depression. In some embodiments, the subject has late-life depression.

In some embodiments, the voxelwise analysis for determining if a subject has or is at risk of developing psychomotor slowing comprises determining at least one topographical pattern within the brain region of interest. In some embodiments, the at least one topographical pattern includes at least one pattern comprising a change in cell number in the brain region of interest.

In some embodiments, the one or more acquired NM-MRI scans for determining if a subject has or is at risk of developing psychomotor slowing are related to the subject's performance on a cognitive task. In some embodiments, the cognitive task assesses catecholamine-related processes. In some embodiments, the catecholamine-related processes comprise dopamine-related processes. In some embodiments, the catecholamine-related processes comprise reward processing. In some embodiments, the one or more acquired NM-MRI scans are related to the subject's performance a gait speed task. In some embodiments, the one or more acquired NM-MRI scans are related to the subject's performance a processing speed task.

In some embodiments, the brain region is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In some embodiments, any one of the methods described herein is used with a second imaging method. In some embodiments, the second imaging method comprises Positron Emission Tomography (PET). In some embodiments, the second imaging method comprises structural MRI. In some embodiments, the second imaging method comprises functional MRI (fMRI). In some embodiments, the second imaging method comprises blood oxygen level dependent (BOLD) fMRI.

### BRIEF DESCRIPTION OF THE FIGURES

The patent or application file contains at least one drawing in color.

FIGS. 1A-B show MRI images. (A) Template of the midbrain in MNI space created by averaging spatially normalized NM-MRI images from all participants. The substantia nigra (SN) is clearly visible as a hyperintense region. (B) A mask of the SN (yellow, an over-inclusive mask to ensure full SN coverage for all participants) and the crus cerebri reference region (cyan) in MNI space was traced on the NM-MRI template and applied to all participants for calculation of contrast-to-noise ratio (Methods).

FIGS. 2A-D show comparisons between cocaine users and control. (A) Diagnostic group differences in NM-MRI signal between cocaine users and controls. Scatterplots showing extracted NM-MRI signal (CNR) averaged within cocaine-use voxels (top panel, defined in C), cocaine-use voxels as defined with leave-one-out (LOO) procedure (middle panel), and the whole SN (bottom panel) in participants divided based on diagnosis. To complement results showing the effect of diagnostic group on NM-MRI signal after adjusting for covariates (B and statistics reported in the text), these scatterplots show diagnostic group differences in the raw, unadjusted NM-MRI signal. (B) Receiver-operating-characteristic curves displaying sensitivity and specificity of the NM-MRI signal in separating diagnostic groups based on signal extracted from cocaine-use voxels (top panel), cocaine-use voxels defined with a leave one out procedure (middle panel), and whole SN (bottom panel). The black line represents NM-MRI signal adjusted for age, head coil, and tobacco use covariates; the gray line represents unadjusted NM-MRI signal. (C) Map of voxels where cocaine users exhibited higher NM-MRI signal than controls (shown in red, robust linear regression, p<0.05 one-sided). This set of voxels was above chance level (p_{corrected}=0.025, permutation test). (D) Unthresholded results of the same analysis showing the t-statistic for the diagnostic group effect for all SN voxels. Voxels where NM-MRI signal was higher in the cocaine users are shown in red and voxels where the signal was lower in cocaine users are shown in blue.

FIG. 3 shows a schematic depicting trafficking of dopamine between the cytosolic, vesicular, and synaptic pools in the striatum and subsequent accumulation of NM in the SN (curved arrow) in health and in cocaine use disorder. Boxes with dashed lines show a schematic detail of the striatal synapse between the gray, pre-synaptic dopamine neuron and the green, post-synaptic striatal neuron. Left: the cytosolic dopamine pool is normally converted to NM and accumulates gradually over the lifespan in the cell bodies of pre-synaptic dopamine neurons within the SN in the midbrain. Right: a theoretical scenario is presented to account for changes observed in cocaine use disorder including the decreased dopamine release observed with PET in prior literature and the increased NM-MRI signal reported here. A decrease in VMAT2, also consistent with PET and postmortem studies, could account for both of these: decreased VMAT2 expression would decrease vesicular dopamine and increase the cytosolic dopamine pool from which NM is synthesized. Please see text for alternative interpretations of the data.

FIG. 4 shows clinical and demographic measures.

FIG. 5 shows demographic and clinical characteristics for studies presented in Example 2.

FIGS. 6A-B show that baseline NM-MRI CNR correlates with gait speed at baseline. (a) Map of SN-VTA voxels where NM-MRI CNR positively correlated (thresholded at P<0.05, voxel level) with a single-task measure of gait speed (green voxels) overlaid on the average NM-MRI CNR image from all subjects. (b) Scatterplot showing the average NM-MRI CNR extracted from the significant voxels in a plotted against gait speed for visualization purposes. These plotted data show a Pearson correlation coefficient of 0.49, although this effect-size estimate is likely inflated given the selection of significant voxels for this effect.

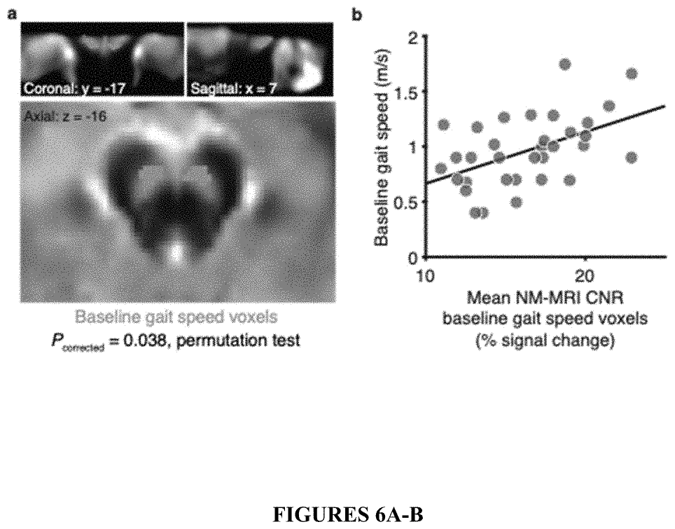

FIGS. 7A-B show that secondary analyses of baseline NM-MRI CNR do not predict changes in gait speed after 3 weeks of L-DOPA treatment in region-of-interest or voxelwise analyses. (a) Scatterplot showing the average NM-MRI CNR extracted from the significant (green) voxels in FIG. 6a plotted against gait speed. These plotted data have a Pearson correlation coefficient of 0.10. (b) Scatterplot showing the average NM-MRI CNR extracted from the voxels where NM-MRI CNR positively correlated with the change in gait speed after 3 weeks of L-DOPA treatment (N=64; thresholded at P<0.05, voxel level). These plotted data have a Pearson correlation coefficient of 0.17.

FIG. 8A-C show that NM-MRI CNR significantly increases after 3 weeks of L-DOPA treatment. (a) Map of SN-VTA voxels where NM-MRI CNR significantly increased after 3 weeks of L-DOPA (thresholded at P<0.05, voxel level; red voxels) overlaid on the average NM-MRI CNR image from all subjects. (b) Histogram showing the average change across subjects in NM-MRI CNR after treatment including all SN-VTA voxels, which is generally shifted to the right of zero (denoting increased NM-MRI CNR). For visualization purposes, heights are proportional to either the number of L-DOPA voxels (N=200; red bars corresponding to voxels in a or the number of Other SN-VTA Voxels (i.e., non-significant voxels; N=1607); e.g., a bar with voxel proportion of 0.2 for L-DOPA voxels corresponds to 40 voxels while a bar with voxel proportion of 0.2 for Other SN-VTA voxels corresponds to 321 voxels. (c) Ladder plot showing the average NM-MRI CNR extracted from the significant (red) voxels in a at baseline (Pre L-DOPA) and after 3 weeks of L-DOPA treatment (Post L-DOPA) for the 6 subjects (each shown in a different color to emphasize consistent increases across each subject).

### DETAILED DESCRIPTION OF THE INVENTION

Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of When the term “about” is used in conjunction with a numerical range, it modifies that range by extending the boundaries above and below the numerical values set forth.

In general, the term “about” is used herein to modify a numerical value above and below the stated value by a variance of 20 percent up or down (higher or lower).

As used herein, the term “subject” refers to a vertebrate animal. In one embodiment, the subject is a mammal or a mammalian species. In one embodiment, the subject is a human. In one embodiment, the subject is a healthy human adult. In other embodiments, the subject is a non-human vertebrate animal, including, without limitation, non-human primates, laboratory animals, livestock, racehorses, domesticated animals, and non-domesticated animals. In one embodiment, the term “human subjects” means a population of healthy human adults.

As used herein, the term “patient” refers to a human or animal.

As used herein, the term “control scan” refers to a baseline scan from a healthy subject without pathology or a baseline scan from the same subject before the subject developed a pathological state. A “control scan” can be utilized for comparison to a subject's scan and determination of pathology in the subject's scan.

Non-Limiting Embodiments

In certain aspects, the invention provides a method for determining dopamine function in a subject, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; determining a NM concentration in the brain region of interest using voxelwise analysis based on the imaging information; and determining the dopamine function based on the NM concentration; wherein the determining of the dopamine function comprises: (1) if the one or more NM-MRI scans has increased NM signal compared to a one or more control scans then dopamine function is increased; or (2) if the one or more NM-MRI scans has decreased NM signal compared to a one or more control scans then dopamine function is decreased.

In some embodiments, the voxelwise analysis comprises determining at least one topographical pattern within the brain region of interest. In some embodiments, the at least one topographical pattern includes at least one pattern comprising a change in cell number in the brain region of interest.

In some embodiments, the one or more acquired NM-MRI scans are related to the subject's performance on a cognitive task. In some embodiments, the cognitive task assesses catecholamine-related processes. In some embodiments, the catecholamine-related processes comprise dopamine- related processes. In some embodiments, the catecholamine-related processes comprise reward processing.

In some embodiments, the brain region is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In some embodiments, the subject has or is suspected of having one or more dopamine function-related disorder. the subject has or is suspected of having schizophrenia spectrum disorders. In some embodiments, the subject has or is suspected of having psychotic illness. In some embodiments, the subject has or is suspected of having addiction disorder. In some embodiments, the subject has or is suspected of having depression. In some embodiments, the subject has or is suspected of having late-life depression. In some embodiments, the subject has or is suspected of having bipolar disorder.

In some embodiments, the subject has or is suspected of having Huntington's disease. In some embodiments, the subject has or is suspected of having Parkinson's disease. In some embodiments, the subject has or is suspected of having one or more movement disorders. In some embodiments, the subject has or is suspected of having psychomotor slowing. In some embodiments, the subject has or is suspected of having one or more neuropsychiatric disorders. In some embodiments, the subject has or is suspected of having a cocaine use disorder.

In certain aspects, the invention provides a method for determining if a subject has or is at risk of developing a neuropsychiatric disorder, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; and determining a NM concentration in the brain region of interest using voxelwise analysis based on the imaging information; wherein the determining if a subject has or is at risk of developing a neuropsychiatric disorder comprises: (1) if the one or more NM-MRI scans has an altered NM signal compared to a one or more control scans without a neuropsychiatric disorder then the subject has or is at risk of developing a neuropsychiatric disorder; or (2) if the one or more NM-MRI scans has a NM signal comparable to the signal of a one or more control scans without a neuropsychiatric disorder then the subject does not have or is not at risk of developing a neuropsychiatric disorder.

In some embodiments, the voxelwise analysis for determining if a subject has or is at risk of developing a neuropsychiatric disorder comprises determining at least one topographical pattern within the brain region of interest. In some embodiments, the at least one topographical pattern includes at least one pattern comprising a change in cell number in the brain region of interest.

In some embodiments, the one or more acquired NM-MRI scans for determining if a subject has or is at risk of developing a neuropsychiatric disorder are related to the subject's performance on a cognitive task. In some embodiments, the cognitive task assesses catecholamine-related processes. In some embodiments, the catecholamine-related processes comprise dopamine-related processes. In some embodiments, the catecholamine-related processes comprise reward processing. In some embodiments, the one or more NM-MRI scans has increased signal compared to a one or more control scans without a neuropsychiatric disorder. In some embodiments, the one or more NM-MRI scans has decreased signal compared to a one or more control scans without a neuropsychiatric disorder.

In some embodiments, the brain region for determining if a subject has or is at risk of developing a neuropsychiatric disorder is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In some embodiments, the neuropsychiatric disorder comprises schizophrenia spectrum disorders. In some embodiments, the neuropsychiatric disorder comprises psychotic illness. In some embodiments, the neuropsychiatric disorder comprises addiction. In some embodiments, the neuropsychiatric disorder comprises depression. In some embodiments, the neuropsychiatric disorder comprises late-life depression. In some embodiments, the neuropsychiatric disorder comprises bipolar disorder.

In some embodiments, the neuropsychiatric disorder comprises Huntington's disease. In some embodiments, the neuropsychiatric disorder comprises psychomotor slowing. In some embodiments, the neuropsychiatric disorder comprises Parkinson's disease. In some embodiments, the neuropsychiatric disorder comprises one or more movement disorders. In some embodiments, the neuropsychiatric disorder comprises cocaine use disorder.

In certain aspects, the invention provides a method for determining if a subject has or is at risk of developing a cognitive disorder, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; and determining a NM concentration in the brain region of interest using voxelwise analysis based on the imaging information; wherein the determining if a subject has or is at risk of developing a cognitive disorder comprises: (1) if the one or more NM-MRI scans has altered signal compared to a one or more control scans without a cognitive disorder then the subject has or is at risk of developing a cognitive disorder; or (2) if the one or more NM-MRI scans has signal comparable to the signal of a one or more control scans without a cognitive disorder then the subject does not have or is not at risk of developing a cognitive disorder.

In some embodiments, the voxelwise analysis for determining if a subject has or is at risk of developing a cognitive disorder comprises determining at least one topographical pattern within the brain region of interest. In some embodiments, the at least one topographical pattern includes at least one pattern comprising a change in cell number in the brain region of interest.

In some embodiments, the one or more acquired NM-MRI scans for determining if a subject has or is at risk of developing a cognitive disorder are related to the subject's performance on a cognitive task. In some embodiments, the cognitive task assesses catecholamine-related processes. In some embodiments, the catecholamine-related processes comprise dopamine-related processes. In some embodiments, the catecholamine-related processes comprise reward processing.

In some embodiments, the one or more NM-MRI scans for determining if a subject has or is at risk of developing a cognitive disorder has increased signal compared to a one or more control scans without a neuropsychiatric disorder. In some embodiments, the one or more NM-MRI scans has decreased signal compared to a one or more control scans without a neuropsychiatric disorder.

In some embodiments, the brain region for determining if a subject has or is at risk of developing a cognitive disorder is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In some embodiments, the cognitive disorder comprises a neurocognitive disorder. In some embodiments, the cognitive disorder comprises memory dysfunction.

In certain aspects, the invention provides a method for determining if a subject has or is at risk of developing an addiction disorder, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; and determining a NM concentration in the brain region of interest using voxelwise analysis based on the imaging information; wherein the determining if a subject has or is at risk of developing an addiction disorder comprises: (1) if the one or more NM-MRI scans has altered NM signal compared to a one or more control scans without an addiction disorder then the subject has or is at risk of developing an addiction disorder; or (2) if the one or more NM-MRI scans has a NM signal comparable to a one or more control scans without addiction disorder then the subject does not have or is not at risk of developing an addiction disorder.

In some embodiments, the voxelwise analysis for determining if a subject has or is at risk of developing an addiction disorder comprises determining at least one topographical pattern within the brain region of interest. In some embodiments, the at least one topographical pattern includes at least one pattern comprising a change in cell number in the brain region of interest.

In some embodiments, the one or more acquired NM-MRI scans for determining if a subject has or is at risk of developing an addiction disorder are related to the subject's performance on a cognitive task. In some embodiments, the cognitive task assesses catecholamine-related processes. In some embodiments, the catecholamine-related processes comprise dopamine-related processes. In some embodiments, the catecholamine-related processes comprise reward processing. In some embodiments, the one or more NM-MRI scans has increased signal compared to a one or more control scans without a neuropsychiatric disorder. In some embodiments, the one or more NM-MRI scans has decreased signal compared to a one or more control scans without a neuropsychiatric disorder.

In some embodiments, the brain region is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In some embodiments, the addiction disorder comprises cocaine use disorder. In some embodiments, the addiction disorder comprises nicotine use disorder. In some embodiments, the addiction disorder comprises alcohol use disorder. In some embodiments, the addiction disorder comprises methamphetamine use disorder. In some embodiments, the addiction disorder comprises opiates use disorder. In some embodiments, the addiction disorder comprises behavioral addictions.

In certain aspects, the invention provides a method of determining if a subject has or is at risk of developing Parkinson's disease, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; and determining a NM concentration in the brain region of interest using voxelwise analysis based on the imaging information; wherein the determining if a subject has or is at risk of developing Parkinson's disease comprises: (1) if the one or more NM-MRI scans has a decreased NM signal compared to a one or more control scans without Parkinson's disease then the subject has or is at risk of developing Parkinson's disease; or (2) if the one or more NM-MRI scans has a NM signal comparable to the signal of a one or more control scans without Parkinson's disease then the subject does not have or is not at risk of developing Parkinson's disease.

In some embodiments, the voxelwise analysis for determining if a subject has or is at risk of developing Parkinson's disease comprises determining at least one topographical pattern within the brain region of interest. In some embodiments, the at least one topographical pattern includes at least one pattern comprising a change in cell number in the brain region of interest.

In some embodiments, the one or more acquired NM-MRI scans for determining if a subject has or is at risk of developing Parkinson's disease are related to the subject's performance on a cognitive task. In some embodiments, the cognitive task assesses catecholamine-related processes. In some embodiments, the catecholamine-related processes comprise dopamine-related processes. In some embodiments, the catecholamine-related processes comprise reward processing.

In some embodiments, the brain region is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In certain aspects, the invention provides a method of determining if a subject has or is at risk of developing psychomotor slowing, the method comprising analyzing one or more Neuromelanin (NM)-Magnetic Resonance Imaging (NM-MRI) scans of the subject's dopamine-associated brain region of interest, wherein the analyzing comprises: receiving imaging information of the brain region of interest; and determining a NM concentration in the brain region of interest using voxelwise analysis based on the imaging information; wherein the determining if a subject has or is at risk of developing psychomotor slowing comprises: (1) if the one or more NM-MRI scans has a decreased NM signal compared to a one or more control scans without psychomotor slowing then the subject has or is at risk of developing psychomotor slowing; or (2) if the one or more NM-MRI scans has a NM signal comparable to the signal of a one or more control scans without psychomotor slowing then the subject does not have or is not at risk of developing psychomotor slowing.

In some embodiments, the subject has depression. In some embodiments, the subject has late-life depression.

In some embodiments, the voxelwise analysis for determining if a subject has or is at risk of developing psychomotor slowing comprises determining at least one topographical pattern within the brain region of interest. In some embodiments, the at least one topographical pattern includes at least one pattern comprising a change in cell number in the brain region of interest.

In some embodiments, the one or more acquired NM-MRI scans for determining if a subject has or is at risk of developing psychomotor slowing are related to the subject's performance on a cognitive task. In some embodiments, the cognitive task assesses catecholamine-related processes. In some embodiments, the catecholamine-related processes comprise dopamine-related processes. In some embodiments, the catecholamine-related processes comprise reward processing. In some embodiments, the one or more acquired NM-MRI scans are related to the subject's performance a gait speed task. In some embodiments, the one or more acquired NM-MRI scans are related to the subject's performance a processing speed task.

In some embodiments, the brain region is the substantia nigra. In some embodiments, the brain region is the ventral substantia nigra. In some embodiments, the brain region is the lateral substantia nigra. In some embodiments, the brain region is the ventrolateral substantia nigra. In some embodiments, the brain region is the substantia nigra pars compacta (SNpc). In some embodiments, the brain region is the substantia nigra pars reticulata (SNpr). In some embodiments, the brain region is the ventral tegmental area (VTA).

In some embodiments, any one of the methods described herein is used with a second imaging method. In some embodiments, the second imaging method comprises Positron Emission Tomography (PET). In some embodiments, the second imaging method comprises structural MRI. In some embodiments, the second imaging method comprises functional MRI (fMRI). In some embodiments, the second imaging method comprises blood oxygen level dependent (BOLD) fMRI.

Conventional MRI does not provide the data needed to predict clinical outcomes in many functional CNS disorders. However, recent methods are being developed to use MRI to detect levels of neuromelanin in the brain. This new technique is expected to provide outcome measures that can predict clinical progression, severity, and response in certain neurologic and psychiatric disorders, including Parkinson's disease, depression, schizophrenia, addiction, and other disorders that involve alterations in the deposition of neuromelanin in the brain or loss of neuromelanin-containing neurons.

Neuromelanin (“NM”) is a black-pigmented product of dopamine and noradrenaline syntheses that accumulates over the lifetime. MRI techniques that can detect neuromelanin can provide insight into the pathophysiology of these disorders. It can also provide useful clinical data in terms of disease progression, clinical severity, and response to treatment.

Imaging of the dopamine and/or norepinephrine system can provide this type of clinically relevant information. Excess dopamine is associated with the development of schizophrenia, symptom severity and treatment response. Low dopamine levels are associated with the development of and symptom severity in Parkinson's disease. In addition, levels of dopamine signaling predict severity of illness and treatment response. Similar results have been shown in depression and other disorders as well.

In particular, previous approaches have been limited to measuring neuromelanin in whole regions or subregions (for instance, across the whole substantia nigra or an anatomically defined subregion within it) and have not capitalized on the high spatial resolution afforded by this neuromelanin-sensitive MRI. This is critical because different populations of neurons within the substantia nigra have distinct function and anatomical connections. In one embodiment, the subject matter disclosed herein relates to the development and validation of a voxelwise method for capitalizing on variability in neuromelanin sensitive MRI signals across voxels, which has the potential to substantially increase the value of neuromelanin-sensitive MRI for clinical applications across neuropsychiatric illnesses. Accordingly, NM-MRI can be used as a marker of integrity or function (e.g., synthesis, transmission, and storage of dopamine) of the dopamine system, relevant to neuropsychiatric disorders affecting this system.

In one embodiment, the subject matter described herein relates to the use of neuromelanin imaging to evaluate the pathological or functional changes in the chatecolamine system that occur in cocaine use disorder and other forms of drug and behavioral addiction. These are conditions where dysregulation of the dopamine system has repeatedly been observed using more direct but invasive imaging measures (e.g., dopamine-receptor positron emission tomography). Neuromelanin-sensitive MRI data may be used as a biomarker for addiction or risk of developing addiction, severity, illness progression, treatment response, and/or clinical outcome. Neuromelanin-sensitive MRI methods meet the need for objective biomarker tracking problematic cocaine use, severity, or risk for its development. Neuromelanin-sensitive MRI can be used as a safe alternative for invasive/radiating imaging measures (e.g., PET). Neuromelanin-sensitive MRI can also be used for monitoring of progression, which currently cannot be done given the risk of repeated exposure to radiation. Neuromelanin-sensitive MRI is non-invasive, cheaper, safer, and easier to acquire in clinical settings. It has substantially increased (5-10-fold) anatomical resolution, which allows for resolving anatomical detail within relevant brain structures.

In one embodiment, the subject matter disclosed herein relates to a neuromelanin-sensitive magnetic resonance imaging (MRI) platform for characterizing disorders linked to dysregulation of the dopamine system, such as cocaine use disorder and other types of addictive behaviors. It uses a validated voxel-wise analysis method to determine topographical patterns within dopaminergic brain regions, such as the substantia nigra, with a high degree of spatial resolution. These patterns can be used to characterize dopaminergic function and cell loss in a variety of neuropsychiatric disorders. This technology is noninvasive and could be used to monitor and predict patient outcomes for various chatecolaminergic disorders including schizophrenia, psychosis, neurodegenerative diseases and addiction-like behaviors.

In some embodiments, neuromelanin MRI signal can be used to determine neuromelanin concentration, dopamine levels in the striatum, substantia nigra blood flow, and severity of psychosis in schizophrenia (Cassidy CM, Zucca A, Girgis R R, Baker S C, Weinstein J J, Sharp M E, Bellei C, Valmadre A, Vanegas N, Kegeles L S, Brucato G, Kang U J, Sulzer D, Zecca L, Abi-Dargham A, Horga G. Neuromelanin-sensitive MRI as a noninvasive proxy measure of dopamine function in the human brain. Proc Natl Acad Sci USA. 2019 Mar. 12; 116(11): pp. 5108-5117.)

In some embodiments, the subject matter disclosed herein relates to a neuromelanin-sensitive magnetic resonance imaging (MRI) platform for characterizing disorders linked to dysregulation of the dopamine system, including a dopamine function-related disorder. In some embodiments, the subject matter disclosed herein relates to the use of a validated voxel-wise analysis method to determine topographical patterns within dopaminergic brain regions, such as the substantia nigra, with a high degree of spatial resolution. In some embodiments, the subject matter disclosed herein relates to a noninvasive and inexpensive method, making it suitable for longitudinal imaging. In some embodiments, the subject matter disclosed herein can be used as an imaging biomarker for monitoring and predicting treatment outcomes for various dopamine function-related disorders (i.e., neurodegenerative diseases, depression, addictive disorders, psychosis, schizophrenia). In some embodiments, the subject matter disclosed herein can be used as a diagnostic biomarker for determining disease severity (e.g., for differential diagnosis across conditions), prognostic indicators of illness progression and/or risk of developing a disorder (genetic, environmental, and clinical risk), and predictive indicators of treatment response (e.g., to aid in individualized treatment selection). In some embodiments, the neuropsychiatric conditions include schizophrenia spectrum disorders, psychotic illness and psychotic symptoms expressed in other conditions (dementia, mood disorders, post-partum syndromes), addiction (cocaine, nicotine, alcohol, methamphetamine, opiates, behavioral addictions), depression (including late-life depression), bipolar disorder, Huntington's disease, psychomotor slowing in aging and other aging-related conditions, Parkinson's disease, and other movement disorders and symptoms (e.g., MSA, PSP, Parkinsonism symptoms, dyskinesia, dystonia).

Non-limiting potential applications for the subject matter disclosed herein also include as an imaging biomarker for drug or behavioral addiction, monitoring treatment outcomes in patients with neuropsychiatric disorders, stratifying patients based on disease severity, predicting the risk of developing addiction (i.e., substance use, behavioral), predicting outcomes of clinical trials, and as a research tool for characterizing in vivo dopamine dysfunction underlying various neuropsychiatric diseases.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Evidence for Dopamine Abnormalities in the Substantia Nigra in Cocaine Addiction Revealed by Neuromelanin-Sensitive MRI

Abstract

Objective: Recent evidence supports the use of neuromelanin-sensitive MRI (NM-MRI) as a novel tool to investigate dopamine function in the human brain. The goal of this study was to investigate the NM-MRI signal in cocaine use disorder, compared to age and sex-matched controls, based on previous imaging studies showing that this disorder is associated with blunted pre-synaptic striatal dopamine.

Methods: NM-MRI and T1-weighted images were acquired from 20 participants with cocaine use disorder and 35 controls. Diagnostic group effects in NM-MRI signal were determined using a voxelwise analysis within the substantia nigra (SN). A subset of 20 cocaine users and 17 controls also underwent functional MRI imaging using the Monetary Incentive Delay task, in order to investigate whether NM-MRI was associated with alterations in reward processing.

Results: Compared to controls, cocaine users showed significantly increased NM-MRI signal in ventrolateral regions of the SN (linear regression; corrected p=0.025, permutation test; area under the receiver-operating-characteristic curve=0.83). Exploratory analyses did not find a significant correlation of NM-MRI signal to activation of the ventral striatum during anticipation of monetary reward.

Conclusions: Given that previous imaging studies show decreased dopamine signaling in the striatum, the finding of increased NM-MRI signal in the SN provides additional insight into the pathophysiology of cocaine use disorder. One interpretation is that cocaine use disorder is associated with a redistribution of dopamine between cytosolic and vesicular pools, leading to increased accumulation of neuromelanin. The study thus suggests that NM-MRI can serve as a practical imaging tool for interrogating the dopamine system in addiction.

Introduction

Alterations of dopamine function have been previously demonstrated in cocaine use disorder using Positron Emission Tomography (PET), including measures of dopamine uptake, receptor density, and dopamine release (1). The reduction of stimulant-induced pre-synaptic dopamine release in cocaine users, measured with PET, is well replicated (1-4) and associated with more refractory symptoms of cocaine use disorder, including relapse (1, 2). However, while PET can provide important insights regarding dopamine signaling in addiction, it is costly and requires significant specialized infrastructure. Further, its use in longitudinal studies and research in younger, at-risk, populations is limited by radioactivity exposure.

Recent work suggests that neuromelanin-sensitive magnetic resonance imaging (NM-MRI) may provide a complementary noninvasive proxy measure of dopamine function and integrity (5, 6). Neuromelanin (NM) is a pigment generated from the conversion of cytosolic dopamine that accumulates gradually over the lifespan in dopamine neurons of the substantia nigra (SN) (7). Neuromelanin is bound to iron, forming paramagnetic complexes that can be imaged using MRI (6, 8, 9). NM-MRI can reliably capture neuromelanin depletion following SN neurodegeneration in Parkinson's disease (6, 10). Critically, this technique can also capture alterations in dopamine function in the absence of neurodegeneration (5, 11), consistent with in vitro evidence that stimulating dopamine synthesis boosts NM synthesis (12, 13).

In particular, NM-MRI signal within a subregion of the substantia nigra is increased in relation to psychosis (5), consistent with PET findings of increased dopamine signaling in psychosis (14). Furthermore, NM-MRI signal correlates directly with both PET measures of pre-synaptic dopamine release and resting blood flow in the midbrain (5). Thus, in one embodiment, the subject matter disclosed herein demonstrates that NM-MRI provides a proxy measure for functional changes in dopaminergic pathways with utility for studying psychiatric disorders without overt neurodegeneration.

Here, NM-MRI was employed for the first time to examine if similar changes could be detected in cocaine use disorder, a disorder involving dopamine dysfunction. To this end, the main analyses herein tested for effects of diagnostic group on NM-MRI signal in the substantia nigra. Without being bound by theory, based on previous PET studies (1, 3), it is thought that cocaine use disorder would be associated with reduced NM-MRI signal. In exploratory analyses, evaluated associations between changes in NM-MRI signal intensity in cocaine use disorder and hemodynamic brain responses during the Monetary Incentive Delay task were evaluated. Activation of the ventral striatum during the anticipation of reward in this task has been shown to provide a robust functional readout of reward processing (15) related to dopamine (16, 17) that is consistently reduced in drug and behavioral addictions (18, 19). Since the ventral striatum receives projections from ventral tegmental area and the dorsomedial SN (20, 21), the relationship between NM-MRI signal in the SN and reward-related activation in ventral striatum was explored.

Methods

Participants

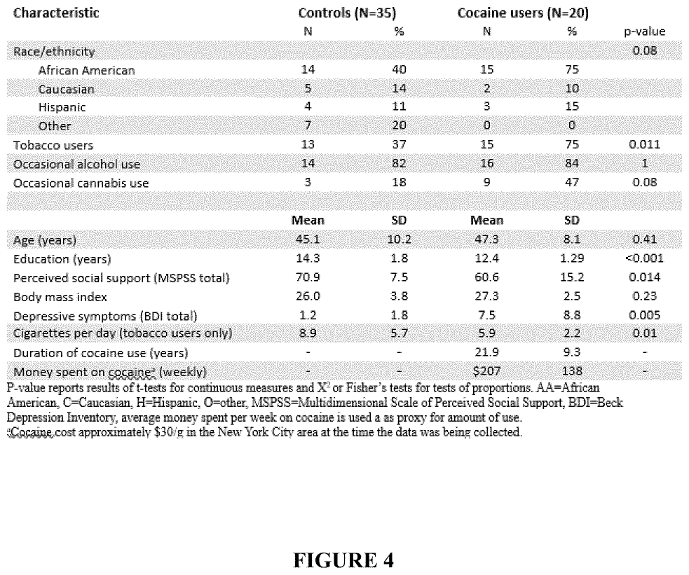

This study was approved by the Institutional Review Board of the New York State Psychiatric Institute. All participants provided written informed consent. The cocaine using participants met DSM-V criteria for moderate to severe cocaine use disorder with no other current Axis I diagnosis or current medical illness. Any other substance use disorder (aside from tobacco and cocaine) was an exclusion criterion. At the time of inclusion, these participants were actively using smoked cocaine, which was verified by urine toxicology. They were required to be abstinent for a minimum of 5 days prior to the scan, which was verified by urine drug testing (performed every other day). Participants refrained from tobacco use for one hour at minimum prior to scanning. A group of tobacco using and non-tobacco using controls was also included. Screening procedures included a physical exam, electrocardiogram, and laboratory tests. All participants were recruited through advertisements and by word-of-mouth. Controls were excluded for: current or past Axis I disorder (except tobacco use disorder), history of neurological disorders, or current major medical illness. In total, 58 males participated in the study. Three participants (1 cocaine user and 2 controls) were excluded due to unusable NM-MRI images (either due to participant motion [showing clearly visible, smearing or banding artifacts affecting the midbrain, n=2] or due to incorrect image-stack placement [n=1]). Thus, a total of 55 participants were retained for analysis: 20 cocaine users and 35 age and sex-matched controls as shown in FIG. 4. All participants completed self-report questionnaires including the Multidimensional Scale of Perceived Social Support (22) and the Beck Depression Inventory (23).

NM-MRI acquisition

Magnetic resonance (MR) images were acquired for all study participants on a GE Healthcare 3T MR750 scanner using a 32-channel, phased-array Nova head coil following methods in prior work (5). For logistical reasons, a few scans (7% of all scans, 4 out of a total of 55) were acquired using an 8-channel Invivo head coil instead. NM-MRI images were acquired using a 2D gradient response echo sequence with magnetization transfer contrast (2D GRE-MT) with the following parameters: repetition time (TR)=260 ms; echo time (TE)=2.68 ms; flip angle=40°; in-plane resolution=0.39×0.39 mm2; partial brain coverage with field of view (FoV)=162×200; matrix=416×512; number of slices=10; slice thickness=3 mm; slice gap=0 mm; magnetization transfer frequency offset=1,200 Hz; number of excitations (NEX)=8; acquisition time=8.04 minutes. The slice-prescription protocol consisted of orienting the image stack along the anterior-commissure—posterior-commissure line and placing the top slice 3 mm below the floor of the third ventricle (for more detail, see (5)). This protocol provided coverage of SN-containing portions of the midbrain and surrounding structures. To support the preprocessing of NM-MRI images (see below), whole-brain, high-resolution T1-weighted structural MRI scans were also acquired using a fast spoiled gradient echo sequence (inversion time=500 ms, TR=6.37 ms, TE=2.59 ms, flip angle=11°, FoV=256x256, number of slices=244, isotropic voxel size=1.0 mm3) or, in some cases, a 3D BRAVO sequence (inversion time=450 ms, ms, TE 3.10 ms, flip angle=12°, FoV=24033 240, number of slices=220, isotropic voxel size=0.8 mm3). Quality of NM-MRI images was visually inspected for artifacts immediately after acquisition, and scans were repeated when necessary, time permitting.

NM-MRI Preprocessing

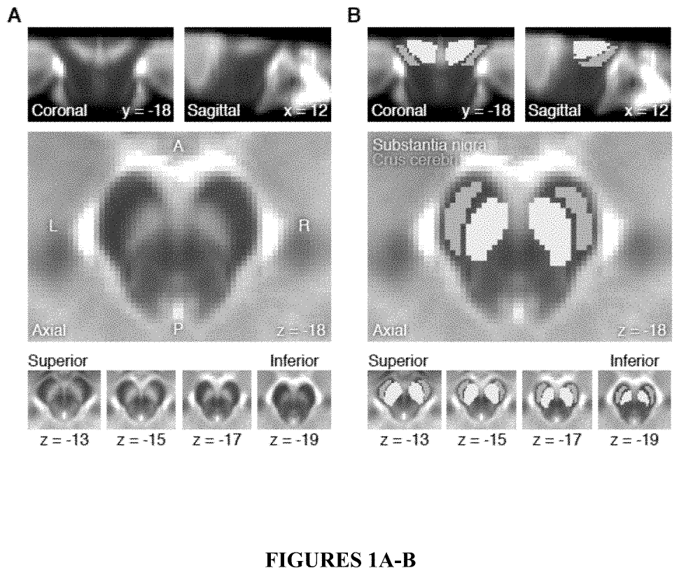

As in prior work (5), NM-MRI scans were preprocessed using SPM12 to allow for voxelwise analyses in standardized MNI space. NM-MRI scans were first coregistered to participants' T1-weighted scans. Tissue segmentation was then performed using the T1-weighted images. NM-MRI scans were normalized to MNI space using DARTEL routines with a gray-and white-matter template generated from all study participants. The resampled voxel size of unsmoothed, normalized NM-MRI scans was 1 mm, isotropic. All images were visually inspected after each preprocessing step. Intensity normalization and spatial smoothing were then performed using custom Matlab (Mathworks) scripts. Contrast-to-noise ratio (CNR) for each participant and voxel v was calculated as the relative difference in NM-MRI signal intensity I from a reference region RR of white matter tracts known to have minimal NM content, the crus cerebri, as: CNR_{V}=(Iᵥ−mode(I_{RR}))/mode(I_{RR}). A template mask of the reference region and of the SN was created by manual tracing on a template NM-MRI image in MNI space (an average of normalized NM-MRI scans from all study participants, see FIG. 1 and previous report for more details (5)). The mode(I_{RR}) was calculated for each participant from a kernel-smoothing-function fit to a histogram of the distribution of all voxels in the mask. The resulting NM-MRI contrast-to-noise ratio maps were then spatially smoothed with a 1-mm full-width-at-half maximum Gaussian kernel.

NM-MRI Analysis

All analyses were carried out in Matlab. Following prior studies (5), the main analysis consisted of a voxelwise analysis of contrast-to-noise ratio values in the SN mask. This approach captures topographic alterations presumably corresponding with functionally distinct SN neuron subpopulations (20) and which previously showed high sensitivity to dopaminergic pathophysiology (5). In particular, the primary voxelwise analysis examined specific differences between cocaine users and controls via a robust linear regression analysis (robustfit function in Matlab) that predicted contrast-to-noise ratio (NM signal) at every voxel v within the SN mask as: CNRᵥ=β₀+β₁·diagnosis+Σᵢ₌₂ⁿβᵢ·nuisance covariate+ϵ, with tobacco use (cigarettes per day), head coil and age as nuisance covariates. Note that correcting for age is critical given the known relationship between age and neuromelanin accumulation (7). As in prior work (5), a group-derived template SN mask was used after censoring participant data points with missing values due to incomplete SN coverage or extreme values (contrast-to-noise ratio<−8% or contrast-to-noise ratio>40%; on average 71±195 voxels or 4% of all SN voxels were censored per subject). To correct for multiple comparisons and again following the prior work (5), the spatial extent of an effect was defined as the number of voxels k (adjacent or nonadjacent) exhibiting diagnostic differences (between cocaine users and controls) in NM signal in either the positive or the negative direction (voxel-level height threshold for t-test of regression coefficient β₁ of p<0.05, one-sided; note that the results remained significant at a more stringent height threshold of p<0.01). Significance testing was then determined based on a permutation test in which diagnosis labels were randomly shuffled with respect to individual maps of NM signal. This provided a measure of spatial extent for each of 10,000 permuted datasets, forming a null distribution against which to calculate the probability of observing the spatial extent k of the effect in the true data by chance. Thus, this test corrects for multiple comparisons by determining whether an effect's spatial extent k was greater than would be expected by chance (p_{corrected}<0.05; 10,000 permutations).

For a more detailed topographical description of the voxelwise effects in the SN, a post-hoc, multiple-linear regression analysis across SN voxels was used to predict the strength of an effect as a function of MNI voxel coordinates in the x (absolute distance from the midline), y, and z directions within the SN mask. For completeness, a region-of-interest analysis was also carried out on the average NM signal across the whole SN mask. This region-of-interest analysis consisted of a robust linear regression analysis including head coil, age, and incomplete SN coverage (yes/no) as nuisance covariates.

The ability of NM-MRI to segregate participants based on diagnostic group was determined by calculating effect size estimates and area under the receiver-operating-characteristic curve based on the mean NM-MRI signal in voxels identified in the primary voxelwise analysis to be relevant to cocaine use disorder (henceforth referred to as “cocaine-use voxels”: voxels showing a diagnosis effect via the primary voxelwise analysis or via a voxelwise analysis following a leave-one-out procedure. The leave-one-out procedure was employed to obtain an measure of effect size unbiased by voxel selection: for a given participant, voxels where the variable of interest was related to NM-MRI signal were first identified in an analysis including all participants except for this (held-out) participant. The mean signal in the held-out participant was then calculated from this set of voxels. This procedure was repeated for all participants so that each participant had an extracted, mean NM-MRI signal value obtained from an analysis that excluded them. Confidence intervals for Cohen's d and f² effect-size measures were determined by bootstrapping.

Partial correlations related clinical measures to NM-MRI signal extracted from cocaine-use voxels, with age and tobacco use as covariates. Partial (nonparametric) Spearman correlation was used because the clinical measures were not normally distributed according to a Lilliefors test at p<0.05.

fMRI methods

fMRI data were collected in 37 of the study participants (20 cocaine users, 17 controls). Blood oxygen level dependent (BOLD) fMRI was acquired while participants completed the Monetary Incentive Delay task. Echo planar images were acquired with the following parameters: repetition time (TR)=1500 ms; echo time (TE)=27 ms; flip angle=60°; in-plane resolution=3.5×3.5 mm²; slice thickness=4 mm; slice gap=1 mm. There were 2 runs each lasting 12.1 minutes. fMRI images were preprocessed using standard methods in SPM12 including slice-time correction, realignment, coregistration to the T1-weighted scans, spatial normalization to standardized MNI space, and smoothing (6 mm full-width at half maximum kernel). The Monetary Incentive Delay task employed was similar to a standard version (24) involving presentation of visual cues (geometric shapes) linked to subsequent receipt of feedback regarding monetary reward ($1 or $5), monetary loss ($1 or $5), or no outcome ($0). The task consisted of 110 trials equally divided into the 5 conditions. Earning money or avoiding losses was probabilistically achieved by having participants make fast key presses following the visual cue. The time available to make a key press was personalized based on participants' motor speed during practice testing. A first-level model included boxcar regressors for all 5 conditions during the anticipation period (defined as the period following button pressing and prior to feedback), the prospect period (following cue presentation and prior to button pressing), and the outcome period (when feedback was delivered). Nuisance regressors included 24 motion parameters (6 motion parameters and their squares, derivatives, and squared derivatives) and session-specific intercepts corresponding to the 2 runs. As in prior work (15), activation during reward anticipation was defined by the contrast between the $5 versus $0 gain conditions. For each participant, the signal from this contrast within a mask of the ventral striatum (from a publicly available functional mask of the striatum //osf.io/jkzwp/) was extracted. The ventral striatum is the brain structure most commonly investigated when using this task (19) and has been shown to provide a robust and reliable readout of reward-related activity during this task (25). To determine relationship to NM-MRI, a linear regression was used to investigate the effect of diagnosis, NM-MRI signal in cocaine-use voxels, and the interaction of diagnosis by NM-MRI signal on anticipatory BOLD activity in the ventral striatum controlling for age and tobacco use.

Results

Effect of Diagnosis on NM-MRI Signal in the Substantia Nigra

A Priori Voxelwise Analysis of Differences Between Cocaine Users and Controls

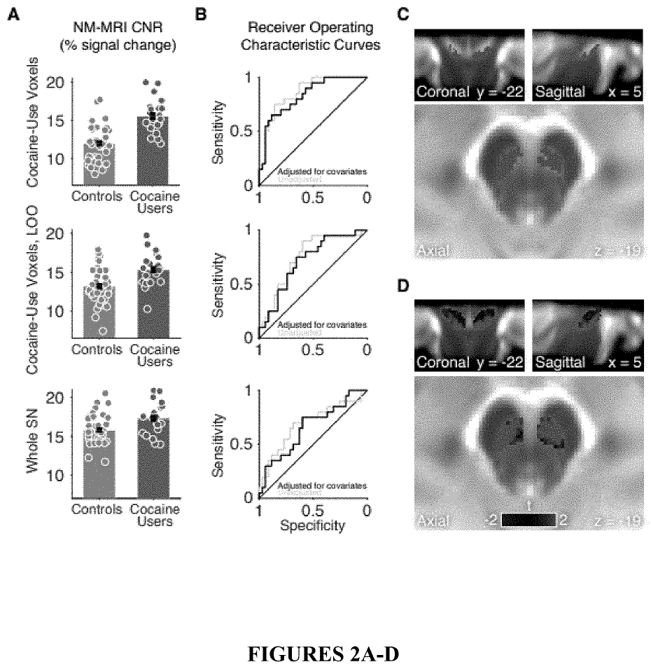

A subset of voxels located mostly ventro-laterally within the SN exhibited significantly increased NM-MRI signal (contrast-to-noise ratio) in cocaine users compared to controls (344 of 1775 voxels at p<0.05, robust linear regression controlling for age, head coil, and cigarettes per day; p_{corrected}=0.025, permutation test; peak voxel MNI coordinates [x, y, z]: 6, −26, −17 mm; see FIG. 2B). In this sample of relatively light smokers, tobacco use was not significantly associated with differences in NM-MRI signal (267 SN voxels exhibited signal that positively correlated with cigarettes per day in the primary linear regression model, P_{corrected}=0.054).

Based on the average NM-MRI signal values extracted from the voxels where cocaine users showed increased NM-MRI signal relative to controls in the voxelwise analysis (cocaine-use voxels, shown in red in FIG. 2B, with extracted values from these voxels shown in FIG. 2A top panel), a diagnosis of cocaine use disorder had a moderate to large effect on NM-MRI signal (Cohen's d=1.34, 95% confidence interval [CI]=0.91-1.90, Cohen's f₂=0.46, 95% CI=0.19-0.95; unbiased leave-one-out Cohen's d=0.77, 95% CI=0.35-1.27, Cohen's f2=0.15, 95% CI=0.02-0.43; all estimates based on NM-MRI signal adjusted for age, head coil, and tobacco use). Diagnostic differences in adjusted NM-MRI signal extracted from cocaine-use voxels remained moderate to large when analyzing subsets of the study sample to address possible confounds (controlling for years of education: Cohen's d=0.76, 95% CI=0.22-1.39, n=38; controlling for depressive symptoms: Cohen's d=0.84, 95% CI=0.31-1.52, n=37; controlling for perceived social support: Cohen's d=1.06, 95% CI=0.52-1.72, n=37; excluding non-tobacco users: Cohen's d=1.05, 95% CI=0.50-1.74, n=28; excluding participants scanned with 8-channel coil: Cohen's d=1.38, CI=0.93-1.97, n=51). Furthermore, most cocaine users could be successfully classified relative to all 35 controls based on adjusted NM-MRI signal extracted from cocaine-use voxels (area under the receiver operating characteristic curve [AUC]=0.83, unbiased leave-one-out AUC=0.71; FIG. 2).

For completeness, NM-MRI signal averaged within the whole SN using a region-of-interest analysis was examined. Here again, cocaine users showed significantly increased NM-MRI signal compared to controls (t49=2.07, p=0.044, Cohen's d=0.62, 95% CI=0.19-1.12, robust linear regression controlling for age, head coil, tobacco use, and incomplete SN coverage; AUC=0.69).

Exploratory analysis of the relationship between NM-MRI signal in substantia nigra and measures of cocaine use severity

It was tested whether the NM-MRI signal extracted from cocaine-use voxels correlated with severity of cocaine use and found no significant correlation with duration of use (p=-0.33 p=0.18) or money spent on cocaine per week (p=-0.08, p=0.74; partial Spearman correlations controlling for age and tobacco use).

Exploratory Analysis of the Relationship Between NM-MRI Signal in Substantia Nigra and Ventral Striatum Response to Reward Anticipation

To investigate the relationship of the NM-MRI findings to dopamine-related circuit dysfunction in cocaine use disorder, fMRI BOLD activation was measured in the ventral striatum during anticipation of monetary reward. As expected, across all participants, BOLD signal was higher in ventral striatum when anticipating reward compared to no reward (t₃₆=2.56, p=0.015, one-sample t-test of [$5-$0] contrast during anticipation). But this reward-related activation in ventral striatum did not differ between the groups (β=0.038, t32=0.72, p=0.48) or correlate with NM-MRI signal in cocaine use voxels across all participants (β3=−0.015, t₃₂=−1.52, p=0.14). There was also no group by NM-MRI signal interaction on reward-related activation in ventral striatum (p=0.24; linear regression controlling for age and tobacco use).

Discussion

Data is presented herein showing increased NM-MRI signal in the SN of individuals with cocaine use disorder. This increase was not present throughout the whole SN but rather predominated in more ventral and lateral SN subregions. Given that the NM-MRI signal reflects the concentration of synthetic melanins in experimental preparations (8) and of NM in postmortem midbrain tissue (5), and that NM accumulation in SN depends on dopamine function (5, 12, 13), these findings suggest that cocaine users exhibit elevated NM concentration in these SN subregions that may be indicative of dopaminergic dysfunction.

The finding of elevated NM signal in cocaine users was surprising given the previous PET studies showing that pre-synaptic dopamine is blunted in cocaine use disorder (1-4). However, this discrepancy provides additional insight into the pathophysiology of dopamine signaling in this disorder. The combination of blunted dopamine release in the striatum with elevated NM in the SN suggests that dopamine is distributed differently in cocaine users compared to controls. Less dopamine concentrated in synaptic vesicles and more dopamine in the cytosolic pool would explain the divergence between PET studies, which estimate dopamine release from vesicles, versus imaging of NM, which accumulates based on the concentration of dopamine in the cytosol (12, 26). If, on the other hand, cocaine use disorder were associated with a global and persistent decrease in dopamine synthesis, a decrease in both the PET and NM-MRI signal would have been expected.

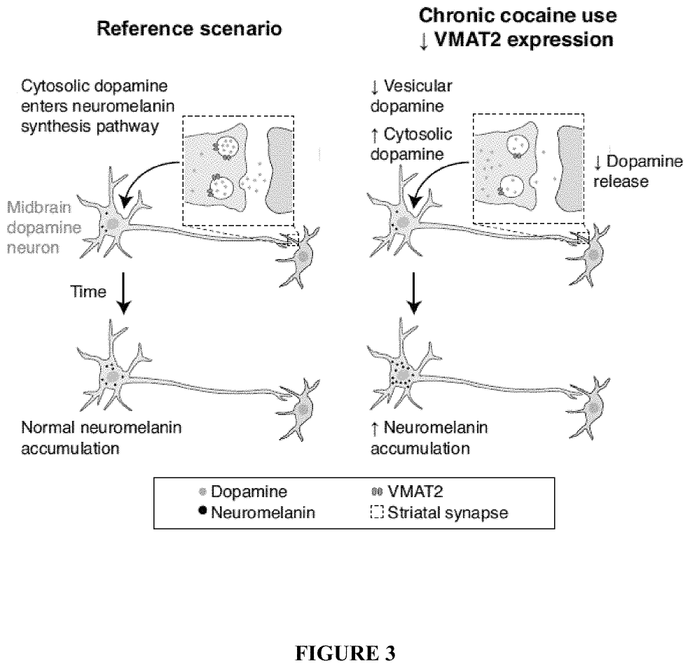

There are a number of previous studies that support the hypothesis that cocaine use disorder involves a redistribution of dopamine between vesicular and cytosolic stores (for graphical depiction of this hypothesis, see FIG. 3). Chronic cocaine exposure is associated with a reduction in vesicular monoamine transporter 2 (VMAT2) expression, which leads to less dopamine in the vesicular pool and more in the cytosolic pool. The reduction in VMAT2 has been shown in nonhuman primates who chronically self-administer cocaine (27) and in human cocaine users (28). Post-mortem human studies also show a reduction of striatal VMAT2 in cocaine users (29-31).

Blunted VMAT2 expression in cocaine use disorder would explain the decrease in pre-synaptic dopamine release seen with PET (1-4) and could also account for the decrease in [18F]DOPA accumulation seen in this population (32), since this likely depends on the radiotracer concentrating in synaptic vesicles (33). Reduced VMAT2 expression has also been shown to correlate with elevated NM formation in the midbrain (12, 34). While cocaine use has been shown to be associated with altered expression of D2 autoreceptors and several other proteins (1, 35), these changes would generally shift both NM accumulation and dopamine release in the same direction. VMAT2 alteration, on the other hand, stands out as a parsimonious explanation for the observed changes occurring in opposing directions. Taken together, these imaging studies suggest that cocaine use is associated with lower dopamine in the vesicular pool and a higher concentration in the cytosolic compartment. However, a study imaging VMAT2 and dopamine release in cocaine users combined with NM-MRI in the midbrain would be needed to confirm the hypothesis. If cocaine use indeed increases cytosolic dopamine, this may pose a risk to neurons because oxidation of dopamine in this compartment forms reactive quinone species (36); however, there is no clear evidence of enhanced dopamine cell death (37) or Parkinson's disease risk (38) in cocaine users.

An alternative interpretation of the main finding is that NM elevation in cocaine users results from repeated episodic surges in dopamine that occurred over the participants' lifetime, which may not be captured by PET. Since NM granules are only removed following cell death (26), and thus serve as a long-term reporter of dopamine function, even a distant history of cocaine use (which may acutely lead to excess dopamine during cocaine consumption) could manifest as a persistent increase in the NM-MRI signal. Future longitudinal studies would be needed to address this possibility.

As an initial test of the functional significance of the findings, it was examined whether NM-MRI signal in cocaine-use voxels within the SN correlated with fMRI response to reward anticipation in the ventral striatum during the Monetary Incentive Delay task, a robust probe of reward system function (15, 19, 25). A significant correlation was not found. This is perhaps unsurprising since the abnormality in cocaine users was not clustered near the “limbic” SN or ventral tegmental area [dorsomedial regions of the over-inclusive SN mask (21)] that send the main projections to ventral striatum. Rather, the topographical analysis showed that group differences predominated in the ventral (or “cognitive”) SN (21), a subregion with prominent projections to the dorsal striatum thought to be involved in cognitive flexibility and other higher-order functions. While PET imaging studies of dopamine function in cocaine users have found consistent evidence of dopaminergic alterations in the dorsal striatum, they have also found pronounced alterations in the ventral striatum. Intriguingly, the observation that cocaine users show an increase in NM-MRI signal in dorsal-striatum-projecting regions of SN but not in ventral-striatum-projecting regions aligns with the previous observation of significant VMAT2 reductions in the dorsal but not the ventral striatum in this population (28, 31). Whatever may underlie this anatomical pattern, it highlights that nigrostriatal circuits sub-serving cognitive functions may be important in cocaine use disorder and that future studies might be better positioned to determine the functional significance of NM-MRI signal change in this disorder by probing higher-order cognitive processes in addition to reward tasks.

The primary limitation of this study is the relatively small, entirely male, sample. However, this first report of NM-MRI in substance use disorders supports the promise of this method for measuring dopamine function in this population. The only previous NM-MRI study to investigate substance use was a preliminary evaluation of the size of the SN area in a small group of patients with psychotic illness. Psychotic patients with comorbid substance use exhibited a larger SN area than non-user patients (39). There is no previous work investigating NM concentration in post-mortem tissue in substance use disorders and this would be an important future direction to provide convergent support for the findings. Further research is needed to address the question of generalization, especially in light of the findings showing a trend-level relationship between NM-MRI and tobacco use (which may well reach significance in a larger sample or in heavier tobacco users). Assuming increased NM signal is due to downregulation of VMAT2 (27, 28), the reported NM-MRI phenotype may be specific to cocaine or other drugs affecting VMAT2 [perhaps including methamphetamine, although its relationship to VMAT2 is less clear (1)]. The absence of significant correlation between NM-MRI signal and duration of cocaine use in the data herein is surprising. Given that NM accumulates over time, it is anticipated that longer duration of use would exaggerate any abnormalities observed in cocaine users. The lack of a significant relationship could, however, be due to the limited range in the duration of use in the sample disclosed herein, as the participants had all been using cocaine for many years. The NM-MRI signal does not reflect a single biological process but could be altered by changes in dopamine synthesis (12), dopamine transfer to vesicles (34), or dopamine cell death (6). Such non-specificity is common to imaging measures (40, 41) and argues for the utility of multimodal studies in triangulating neurobiological mechanisms, as the findings herein can be interpreted in light of previous PET imaging reports. While interpretation of the NM-MRI results is simplified by the absence of enhanced dopamine cell death in cocaine users (37), interpretation of NM-MRI results in disorders showing substantial cell death combined with altered NM accumulation may be more challenging.

Here, NM-MRI evidence has been presented for abnormal NM accumulation in cocaine users, an indirect indication of dopamine dysfunction consistent with prior work. The subject matter disclosed herein thus positions NM-MRI as a promising research tool for addiction and supports its development as a candidate biomarker for stimulant use disorders. Given the central role of dopamine in addiction and the ease of NM-MRI data acquisition, this method has the potential to advance the understanding of dopamine alterations in addiction, particularly as it affords the opportunity to study younger, at-risk populations and describe longitudinal trajectories of dopamine alterations, which have been challenging to study using PET.

References for Example 1

1. Ashok A H, Mizuno Y, Volkow N D, Howes O D. Association of Stimulant Use With Dopaminergic Alterations in Users of Cocaine, Amphetamine, or Methamphetamine: A Systematic Review and Meta-analysis. JAMA Psychiatry. 2017;74:511-519.

2. Martinez D, Carpenter KM, Liu F, Slifstein M, Broft A, Friedman A C, Kumar D, Van Heertum R, Kleber H D, Nunes E. Imaging dopamine transmission in cocaine dependence: link between neurochemistry and response to treatment. Am J Psychiatry. 2011;168:634-641.

3. Martinez D, Narendran R, Foltin R W, Slifstein M, Hwang D R, Broft A, Huang Y, Cooper T B, Fischman M W, Kleber H D, Laruelle M. Amphetamine-induced dopamine release: markedly blunted in cocaine dependence and predictive of the choice to self-administer cocaine. Am J Psychiatry. 2007;164:622-629.

4. Volkow N D, Wang G J, Fowler J S, Logan J, Gatley S J, Hitzemann R, Chen A D, Dewey S L, Pappas N. Decreased striatal dopaminergic responsiveness in detoxified cocaine-dependent subjects. Nature. 1997;386:830-833.

5. Cassidy C M, Zucca F A, Girgis R R, Baker S C, Weinstein J J, Sharp M E, Bellei C, Valmadre A, Vanegas N, Kegeles L S, Brucato G, Jung Kang U, Sulzer D, Zecca L, Abi-Dargham A, Horga G. Neuromelanin-sensitive MRI as a noninvasive proxy measure of dopamine function in the human brain. Proc Natl Acad Sci U S A. 2019;116:5108-5117.

6. Sulzer D, Cassidy C, Horga G, Kang U J , Fahn S, Casella L, Pezzoli G, Langley J, Hu X P, Zucca F A, Isaias I U, Zecca L. Neuromelanin detection by magnetic resonance imaging (MRI) and its promise as a biomarker for Parkinson's disease. NPJ Parkinsons Dis. 2018;4:11.

7. Zecca L, Fariello R, Riederer P, Sulzer D, Gatti A, Tampellini D. The absolute concentration of nigral neuromelanin, assayed by a new sensitive method, increases throughout the life and is dramatically decreased in Parkinson's disease. FEBS Lett. 2002;510:216-220.

8. Trujillo P, Summers P E, Ferrari E, Zucca F A, Sturini M, Mainardi L T, Cerutti S, Smith A K, Smith S A, Zecca L, Costa A. Contrast mechanisms associated with neuromelanin-MRI. Magn Reson Med. 2017;78:1790-1800.

9. Zecca L, Bellei C, Costi P, Albertini A, Monzani E, Casella L, Gallorini M, Bergamaschi L, Moscatelli A, Turro N J, Eisner M, Crippa P R, Ito S, Wakamatsu K, Bush W D, Ward W C, Simon J D, Zucca F A. New melanic pigments in the human brain that accumulate in aging and block environmental toxic metals. Proc Natl Acad Sci USA. 2008;105:17567-17572.

10. Martin-Bastida A, Lao-Kaim N P, Roussakis A A, Searle G E, Xing Y, Gunn R N, Schwarz S T, Barker R A, Auer D P, Piccini P. Relationship between neuromelanin and dopamine terminals within the Parkinson's nigrostriatal system. Brain. 2019;142:2023-2036.

11. Watanabe Y, Tanaka H, Tsukabe A, Kunitomi Y, Nishizawa M, Hashimoto R, Yamamori H, Fujimoto M, Fukunaga M, Tomiyama N. Neuromelanin magnetic resonance imaging reveals increased dopaminergic neuron activity in the substantia nigra of patients with schizophrenia. PLoS One. 2014;9:e104619.

12. Sulzer D, Bogulaysky J, Larsen K E, Behr G, Karatekin E, Kleinman M R, Turro N, Krantz D, Edwards R H, Greene L A, Zecca L. Neuromelanin biosynthesis is driven by excess cytosolic catecholamines not accumulated by synaptic vesicles. Proc Natl Acad Sci USA. 2000;97:11869-11874.

13. Cebrian C, Zucca F A, Mauri P, Steinbeck J A, Studer L, Scherzer C R, Kanter E, Budhu S, Mandelbaum J, Vonsattel J P, Zecca L, Loike J D, Sulzer D. MHC-I expression renders catecholaminergic neurons susceptible to T-cell-mediated degeneration. Nat Commun. 2014;5:3633.

14. McCutcheon R A, Abi-Dargham A, Howes O D. Schizophrenia, Dopamine and the Striatum: From Biology to Symptoms. Trends Neurosci. 2019;42:205-220.

15. Oldham S, Murawski C, Fornito A, Youssef G, Yucel M, Lorenzetti V. The anticipation and outcome phases of reward and loss processing: A neuroimaging meta-analysis of the monetary incentive delay task. Hum Brain Mapp. 2018;39:3398-3418.

16. Urban N B, Slifstein M, Meda S, Xu X, Ayoub R, Medina O, Pearlson G D, Krystal J H, Abi-Dargham A. Imaging human reward processing with positron emission tomography and functional magnetic resonance imaging. Psychopharmacology (Berl). 2012;221:67-77.

17. Schott B H, Minuzzi L, Krebs R M, Elmenhorst D, Lang M, Winz O H, Seidenbecher C I, Coenen H H, Heinze H J, Zilles K, Duzel E, Bauer A. Mesolimbic functional magnetic resonance imaging activations during reward anticipation correlate with reward-related ventral striatal dopamine release. J Neurosci. 2008;28:14311-14319.

18. Luijten M, Schellekens A F, Kuhn S, Machielse M W, Sescousse G. Disruption of Reward Processing in Addiction : An Image-Based Meta-analysis of Functional Magnetic Resonance Imaging Studies. JAMA Psychiatry. 2017;74:387-398.

19. Balodis I M, Potenza M N. Anticipatory reward processing in addicted populations: a focus on the monetary incentive delay task. Biol Psychiatry. 2015;77:434-444.

20. Weinstein J J, Chohan M O, Slifstein M, Kegeles L S, Moore H, Abi-Dargham A. Pathway-Specific Dopamine Abnormalities in Schizophrenia. Biol Psychiatry. 2017;81:31-42.

21. Zhang Y, Larcher K M, Misic B, Dagher A. Anatomical and functional organization of the human substantia nigra and its connections. Elife. 2017;6.

22. Zimet G D, Dahlem N W, Zimet S G, Farley G K. The multidimensional scale of perceived social support. Journal of Personality Assessment. 1988;52:30-41.

23. Beck A T, Steer R A, Ball R, Ranieri W. Comparison of Beck Depression Inventories -IA and -II in psychiatric outpatients. J Pers Assess. 1996;67:588-597.

24. Andrews M M, Meda S A, Thomas A D, Potenza M N, Krystal JH, Worhunsky P, Stevens M C, O'Malley S, Book G A, Reynolds B, Pearlson G D. Individuals family history positive for alcoholism show functional magnetic resonance imaging differences in reward sensitivity that are related to impulsivity factors. Biol Psychiatry. 2011;69:675-683.

25. Wu C C, Samanez-Larkin G R, Katovich K, Knutson B. Affective traits link to reliable neural markers of incentive anticipation. Neuroimage. 2014;84:279-289.

26. Zucca F A, Basso E, Cupaioli F A, Ferrari E, Sulzer D, Casella L, Zecca L. Neuromelanin of the human substantia nigra: an update. Neurotox Res. 2014;25:13-23.

27. Narendran R, Jedema HP, Lopresti B J, Mason N S, Himes M L, Bradberry C W. Decreased vesicular monoamine transporter type 2 availability in the striatum following chronic cocaine self-administration in nonhuman primates. Biol Psychiatry. 2015;77:488-492.

28. Narendran R, Lopresti B J, Martinez D, Mason N S, Himes M, May M A, Daley D C, Price J C, Mathis C A, Frankle W G. In vivo evidence for low striatal vesicular monoamine transporter 2 (VMAT2) availability in cocaine abusers. Am J Psychiatry. 2012;169:55-63.

29. Little K Y, Krolewski D M, Zhang L, Cassin B J. Loss of striatal vesicular monoamine transporter protein (VMAT2) in human cocaine users. Am J Psychiatry. 2003;160:47-55.

30. Little K Y, Zhang L, Desmond T, Frey K A, Dalack G W, Cassin B J. Striatal dopaminergic abnormalities in human cocaine users. Am J Psychiatry. 1999;156:238-245.

31. Wilson J M, Levey A I, Bergeron C, Kalasinsky K, Ang L, Peretti F, Adams V I, Smialek J, Anderson W R, Shannak K, Deck J, Niznik H B, Kish S J. Striatal dopamine, dopamine transporter, and vesicular monoamine transporter in chronic cocaine users. Ann Neurol. 1996;40:428-439.

32. Wu J C, Bell K, Najafi A, Widmark C, Keator D, Tang C, Klein E, Bunney B G, Fallon J, Bunney W E. Decreasing striatal 6-FDOPA uptake with increasing duration of cocaine withdrawal. Neuropsychopharmacology. 1997;17:402-409.

33. Kumakura Y, Cumming P. PET studies of cerebral levodopa metabolism: a review of clinical findings and modeling approaches. Neuroscientist. 2009;15:635-650.

34. Liang C L, Nelson O, Yazdani U, Pasbakhsh P, German DC. Inverse relationship between the contents of neuromelanin pigment and the vesicular monoamine transporter-2: human midbrain dopamine neurons. J Comp Neurol. 2004;473:97-106.

35. Worhunsky P D, Matuskey D, Gallezot J D, Gaiser E C, Nabulsi N, Angarita G A, Calhoun V D, Malison R T, Potenza M N, Carson R E. Regional and source-based patterns of [(11)C]-(+)-PHNO binding potential reveal concurrent alterations in dopamine D2 and D3 receptor availability in cocaine-use disorder. Neuroimage. 2017;148:343-351.

36. Segura-Aguilar J, Paris I, Munoz P, Ferrari E, Zecca L, Zucca F A. Protective and toxic roles of dopamine in Parkinson's disease. J Neurochem. 2014;129:898-915.

37. Bennett B A, Hyde C E, Pecora J R, Clodfelter J E. Long-term cocaine administration is not neurotoxic to cultured fetal mesencephalic dopamine neurons. Neurosci Lett. 1993;153:210-214.

38. Asser A, Taba P. Psychostimulants and movement disorders. Front Neurol. 2015;6:75.

39. Tavares M, Reimao S, Chendo I, Carvalho M, Levy P, Nunes R G. Neuromelanin magnetic resonance imaging of the substantia nigra in first episode psychosis patients consumers of illicit substances. Schizophr Res. 2018;197:620-621.

40. Guo N, Hwang D R, Lo E S, Huang Y Y, Laruelle M, Abi-Dargham A. Dopamine depletion and in vivo binding of PET D1 receptor radioligands: implications for imaging studies in schizophrenia. Neuropsychopharmacology. 2003;28:1703-1711.

41. Logothetis N K. What we can do and what we cannot do with fMRI. Nature. 2008;453:869-878.

### Example 2

### Association Between Neuromelanin-Sensitive MRI Signal and Psychomotor Slowing in Late-Life Depression

Abstract

Late-life depression (LLD) is a prevalent and disabling condition in older adults that is often accompanied by slowed processing and gait speed. These symptoms are related to impaired dopamine function and sometimes remedied by levodopa (L-DOPA). In this study, 33 older adults with LLD were recruited to determine the association between a proxy measure of dopamine function—neuromelanin-sensitive magnetic resonance imaging (NM-MRI)—and baseline slowing measured by the Digit Symbol test and a gait speed paradigm. In secondary analyses, the ability of NM-MRI to predict L-DOPA treatment response in a subset of these patients (N=15) who received 3 weeks of L-DOPA was also assessed. A further subset of these patients (N=6) were scanned with NM-MRI at baseline and after treatment to evaluate the effects of L-DOPA treatment on the NM-MRI signal. It was found that lower baseline NM-MRI correlated with slower baseline gait speed (346 of 1,807 substantia nigra-ventral tegmental area (SN-VTA) voxels, Pcorrected=0.038), particularly in the more medial, anterior, and dorsal SN-VTA. Secondary analyses failed to show an association between baseline NM-MRI and treatment-related changes in gait speed, processing speed, or depression severity (all P_{corrected}>0.361); evidence of increases in the NM-MRI signal 3 weeks post-treatment with L-DOPA compared to baseline was found (200 of 1,807 SN-VTA voxels; P_{corrected}=0.046). Overall, the findings indicate that NM-MRI is sensitive to variability in gait speed in patients with LDD, suggesting this non-invasive MRI measure may provide a promising marker for dopamine-related psychomotor slowing in geriatric neuropsychiatry.

Introduction

Late life depression (LLD) is a prevalent and disabling condition among older adults that is often recurrent, can become chronic, and is frequently non-responsive to antidepressant medication (1-4). Motivational deficits, slowed processing speed, and gait impairments are prominent aspects of the LLD phenotype and suggest dopaminergic dysfunction may play a key pathophysiologic role (5-7). These features are negative prognostic factors for antidepressant treatment (8) and more broadly portend adverse health outcomes, including death (9, 10). Recent work suggests that carbidopa/levodopa (L-DOPA) monotherapy significantly improves processing speed, gait speed, and depressive symptoms in depressed older adults by increasing dopamine availability in selected striatal subregions (11). However, LLD is a heterogeneous and etiologically complex disorder, suggesting the need for non-invasive and scalable methods to identify dopamine-deficient individuals and personalize their treatment. As a first step in this direction, here the ability of neuromelanin-sensitive magnetic resonance imaging (NM-MRI) to capture dopamine-related phenotypes in LDD was tested, particularly psychomotor slowing.

Psychomotor slowing is of great clinical importance to LDD and it has been linked to dopamine function. In LLD, decreased processing speed predicts poorer acute response to antidepressants (8) and higher risk for dementia (12), while slowed gait increases the risk of falls (13), disability (14), and mortality (6). Psychomotor slowing in older individuals is thought to stem at least in part from decreases in dopamine transmission with aging (15-17), consistent with human and preclinical work linking mesostriatal dopaminergic transmission to gait speed (18, 19). Given this link, the presence of psychomotor slowing may indicate an underlying dopaminergic deficit that could be central to the pathophysiology of LDD (7), and which could possibly be remediated via pro-dopaminergic treatments such as L-DOPA. Indeed, previous work showed that, in LLD individuals with slowed gait speed, L-DOPA monotherapy can ameliorate psychomotor slowing and depressive symptoms by normalizing mesostriatal dopamine transmission (11). While these results are encouraging, slowed gait speed is an indirect and unspecific marker of dopamine deficits, suggesting that more direct measures like NM-MRI could optimize the selection of LDD patients who may benefit most from L-DOPA treatment.

NM-MRI is a noninvasive imaging technique that enables visualization of neuromelanin (NM) concentration in NM-rich regions (20, 21). NM is a product of dopamine metabolism that accumulates in the dopaminergic neurons of the substantia nigra (SN) (22-25). NM-MRI imaging of the SN was recently validated as a marker of dopamine function, with the NM-MRI signal correlating with positron emission tomography (PET) measures of dopamine release capacity in the striatum, and capturing dopamine dysfunctions associated with psychiatric illness (20). NM-MRI is therefore uniquely suited as a potential biomarker for treatment selection in patients with dopamine dysfunction, including at least some LDD patients, and one that could be broadly adopted given its non-invasiveness, cost-effectiveness, and lack of ionizing radiation.

The goal of the present study was to determine the suitability of NM-MRI as a potential biomarker for psychomotor slowing and to begin testing its ability to predict and monitor of L-DOPA treatment response in LLD. Without being bound by theory, it is thought that individuals with slower processing and those with slower gait would exhibit lower dopamine function as measured by NM-MRI. Furthermore, in a secondary analysis in a small sample, the ability of NM-MRI to predict the improvement of psychomotor slowing after L-DOPA treatment was investigated. In an analysis in a further subset of patients, the sensitivity of NM-MRI to capture longitudinal changes in dopamine function associated with L-DOPA treatment was also investigated.

Methods and Materials

Subjects

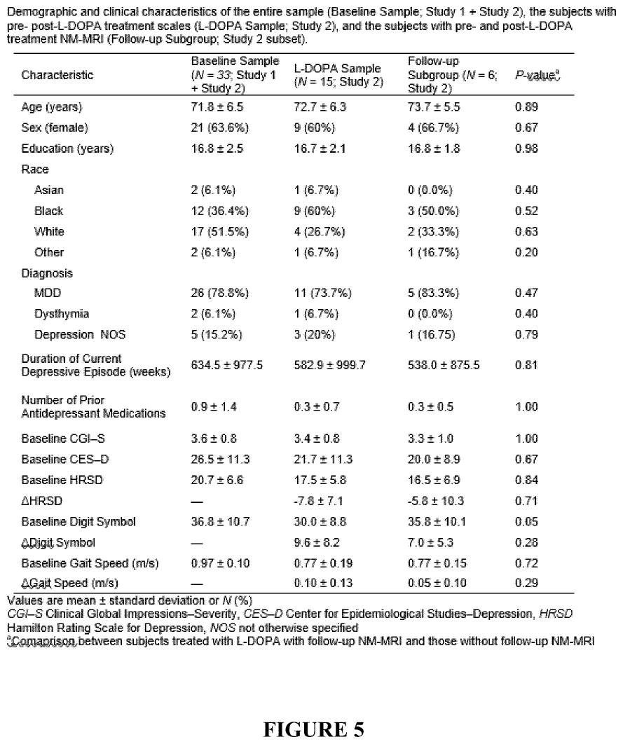

The studies described were conducted in the Adult and Late Life Depression Research Clinic at the New York State Psychiatric Institute (NYSPI) and were approved by the NYSPI Institutional Review Board. The research program on LLD encompasses numerous therapeutic and pathophysiologic studies. In order to increase the sample size, data was aggregated from two studies having similar selection criteria and utilizing the same NM-MRI sequence. The first study (N=18; Study 1) was an antidepressant treatment trial, from which only the baseline data was used. A second study (N=15; Study 2) was an open-label L-DOPA trial, from which the baseline and post-treatment data was used (pre-post L-DOPA dataset). Of these 15 individuals, follow-up NM-MRI data after receiving L-DOPA was collected in 6. See FIG. 5 for further depiction of the sample included in the analyses. All subjects (N=33; Study 1+Study 2) were adult outpatients aged≥60 years who were diagnosed with Diagnostic and Statistical Manual 5 major depressive disorder, dysthymia, or depression not otherwise specified, and had a minimum depressive symptom score on a standardized scale (Hamilton Rating Scale for Depression [HRSD]≥16 or Center for Epidemiologic Studies-Depression Rating Scale≥10). Subjects who exhibited substance abuse or dependence, were diagnosed with a psychotic disorder, bipolar disorder, or probable dementia, had a Mini Mental Status Examination score≤24, an HRSD suicide item>2, or a Clinical Global Impressions-Severity score of 7 at baseline were all excluded. Subjects with an acute or severe medical illness, mobility limiting osteoarthritis or joint disease, a contraindication to MRI, or who had been treated within the past 4 weeks with psychotropic or other medications known to affect dopamine were excluded as well.

Assessments

Processing speed was assessed using the Digit Symbol test from the Wechsler Adult Intelligence Scale-III (26). Gait speed was measured in m/s as a single task in which study participants walked at their usual or normal speed on a 15-foot walking course. Two trials were completed, and the final gait speed measurement was recorded as the average of these two trials. Depression severity was assessed using the 24-item HRSD.

Study 1 Design

Assessments and MRI data were obtained at baseline, prior to beginning antidepressant treatment (N=18). Further details can be found at clinicaltrials.gov/ct2/show/NCT01931202.

Study 2 Design

Inclusion in this study also required decreased gait speed (defined as average walking speed over 15′ course<1 m/s). Assessments and MRI data were obtained at baseline, prior to beginning L-DOPA treatment (N=15). After their MRI scan, subjects began taking 37.5 mg carbidopa/150 mg levodopa once daily (9 am). After one week at this dosage, subjects were instructed to take 37.5 mg carbidopa/150 mg levodopa twice daily (9 am and 5 pm). For the third week of treatment, subjects took 37.5 mg carbidopa/150 mg levodopa three times daily (9 am, 12 pm, and 5 pm). Participants were instructed to maintain the same timing of doses throughout the study as described above. A subset of these participants (N=6) had a post-treatment MRI scan after a Week 3 visit when post-treatment assessments were performed. Please refer to the previously published main outcome manuscript for a full description of study procedures (11); further details can be found at clinicaltrials.gov/ct2/show/NCT02744391. Processing and gait speed were assessed at baseline and then weekly during L-DOPA treatment (i.e., Weeks 0-3). Assessments were performed at approximately 1 pm to control for time of day effects and the duration since the last morning L-DOPA dose (anticipated to be 4 hours). HRSD was also performed at Week 0 and Week 3. Changes in processing speed, gait speed, and HRSD were taken as the difference between Week 3 and Week 0.

Magnetic Resonance Imaging

Magnetic resonance images of the brain were acquired for all participants on a GE MR750 3.0T scanner using a 32-channel phased-array Nova head-coil. NM-MRI data were acquired with a 2D gradient-recalled echo sequence with magnetization transfer contrast (2D GRE-MT) with the following parameters (20): repetition time (TR)=260 ms; echo time (TE)=2.68 ms; flip angle=40°; in-plane resolution=0.39×0.39 mm2; partial brain coverage with field of view (FoV)=162×200; matrix=416×512; number of slices=10; slice thickness=3 mm; slice gap=0 mm; magnetization transfer frequency offset=1,200 Hz; number of excitations (NEX)=8; acquisition time=8.04 min. The slice-prescription protocol consisted of orienting the image stack along the anterior-commissure—posterior-commissure line and placing the top slice 3 mm below the floor of the third ventricle, viewed on a sagittal plane in the middle of the brain. This protocol provided coverage of SN-containing portions of the midbrain (and cortical and subcortical structures surrounding the brainstem) with high in-plane spatial resolution using a short scan easy to tolerate by clinical populations. For preprocessing of the NM-MRI data, a whole-brain, high-resolution T1-weighted 3D BRAVO structural MRI scan was acquired with the following parameters: inversion time=450 ms, TR=7.85 ms, TE=3.10 ms, flip angle=12°, FoV=240×240, matrix=300×300, number of slices=220, isotropic voxel size=0.8 mm3).

NM-MRI data were preprocessed using a pipeline combing SPM and ANTs, previously shown to achieve high test-retest reliability (27). The pipeline consisted of the following steps: (1) brain extraction of the T1w image using ‘antsBrainExtraction.sh’; (2) spatial normalization of the brain-extracted T1w image to MNI space using ‘antsRegistrationSyN.sh’ (rigid+affine+deformable syn); (3) coregistration of the NM-MRI image to the T1w image using ‘antsRegistrationSyN.sh’ (rigid); (4) spatial normalization of the NM-MRI images to MNI space by a single-step transformation combing the transformations estimated in steps (2) and (3) using ‘antsApplyTransforms’; (5) resampling of the spatially-normalized NM-MRI image to 1 mm isotropic resolution using ‘ResampleImage’; (6) spatial smoothing of the spatially-normalized NM-MRI image with a 1 mm full-width-at-half-maximum Gaussian kernel using ‘SPM-Smooth’. The preprocessed NM-MRI images were then used to estimate NM-MRI contrast ratio (CNR) maps. NM-MRI CNR at each voxel was calculated as the percent signal difference in NM-MRI signal intensity at a given voxel (IV) from the signal intensity in the crus cerebri (ICC), a region of white matter tracts known to have minimal NM content as: CNR_{V}={[I_{V}−mode(I_{CC})]/mode(I_{CC})}*100. Where mode(ICC) was calculated for each participant from a kernel-smoothing-function fit of a histogram of all voxels in the CC mask (20).

Statistical Analysis

The a priori analysis tested the hypothesis that lower baseline NM-MRI CNR would correlate with slower psychomotor variables (Digit Symbol and gait speed; N=33; Study 1+Study 2). In a secondary analysis we investigated if baseline NM-MRI CNR would predict L-DOPA-induced improvements (speeding) of these psychomotor variables (N=15; Study 2). These effects were tested within the substantia nigra-ventral tegmental area (SN-VTA) complex using a voxelwise analysis approach validated in Cassidy et al. (20). Briefly, this method uses robust linear regression analyses and tests for significance of regression coefficients using permutation tests. The linear model used to test the a priori hypothesis (model 1) was: CNR_{V}=β₀+β₁·gait speed+β₂·Digit Symbol score+β₃·HRSD+β₄·age+β₅·gender+β₆·education, with β₁₋₃ being the variables of interest and β₄₋₆ covariates of no-interest. The linear model for the secondary analysis (model 2) was: CNR_{V}=β₀+B₁·Δgait speed+β₂ΔDigit Symbol score+β₃ ·ΔHRSD+β₄ ·gait speed+β₅·Digit Symbol score+β₆·HRSD+β₇·age+β₈·gender+β₉·education, with β₁₋₃ being the variables of interest and β₄₋₉ covariates of no-interest. The inclusion of all variables of interest in one model provides greater specificity of effects while also providing a more conservative test that guards against false positives by adjusting the degrees of freedom in t-tests of regression coefficients (28). The number of voxels showing a significant effect was determined to be significant through permutation testing, wherein 10,000 iterations of random permutations of the variables of interest were run while keeping the covariates of no-interest constant—see Cassidy et al. for further details (20). This voxelwise permutation-test corrects for multiple comparisons across voxels and provides adequate protection against false positives, similar to methods used in functional-MRI studies (29).

In an exploratory analysis, we also investigated if changes in NM-MRI CNR can be detected after 3 weeks of L-DOPA treatment (N=6; subset from Study 2). A similar voxelwise analysis approach was used, except it used a non-parametric, sign-rank test comparing pre- and post-L-DOPA treatment NM-MRI CNR values. The number of voxels showing a significant effect was determined to be significant through a permutation test in which the null distribution was derived by 10,000 iterations of random assignment of the pre- and post-L-DOPA treatment labels for each subject (i.e., 50% chance for a subject's pre-L-DOPA treatment NM-MRI CNR value to be assigned as their post-L-DOPA treatment value, with their post-L-DOPA treatment value being assigned as their pre-L-DOPA treatment value).

A priori power analyses using effect sizes comparing baseline gait speed and dopamine function measure by PET (19) demonstrated 85% power to detect an effect in the baseline sample of 33 subjects (two-tailed, α=0.05) but only 50% power in the L-DOPA sample of 15 subjects. Thus, the analyses in the former sample (model 1) were sufficiently powered as the a priori test. No additional corrections were implemented across a priori and secondary tests given the exploratory nature of the latter, which are presented for completeness and descriptive purposes.

To rule out potential selection bias in the follow-up NM-MRI subset from Study 2, Pearson chi-square tests or Mann—Whitney U tests were used to compare demographic and clinical characteristics between the participants in Study 2 who either received a follow-up NM-MRI scan after 3 weeks of L-DOPA treatment (N=6) and those who did not receive a follow-up NM-MRI scan after treatment (N=9).

Results

Sample Characteristics

Clinical and demographic characteristics of the sample are provided in FIG. 5; for all 33 subjects, mean age was 71.8±6.5 years, 63.6% were female, mean education was 16.8±2.5 years, mean gait speed was 0.97±0.32 m/s, mean Digit Symbol score was 36.8±10.7, and mean HRSD was 20.7±6.6. No significant differences were observed between subjects in Study 2 with a follow-up NM-MRI scan and those without a follow-up NM-MRI scan.

Baseline Gait Speed is Associated with Baseline NM-MRI

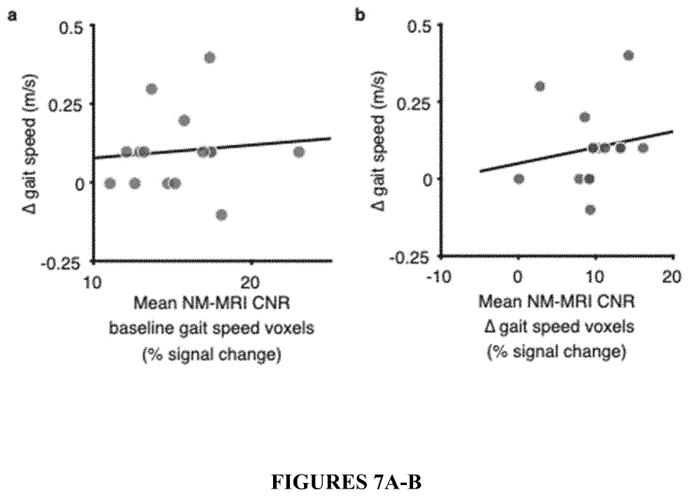

Without being bound by theory, an a priori hypothesis was investigated that individuals with slower processing and those with slower gait would exhibit lower dopamine function as measured by NM-MRI in 33 patients with LLD (Study 1+Study 2). A voxelwise linear regression model (model 1) predicted NM-MRI CNR within the SN-VTA mask as a function of gait speed, Digit Symbol score, and HRSD, with age, gender, and education as covariates. This revealed a set of SN-VTA voxels in which NM-MRI CNR correlated positively with gait speed (346 of 1,807 SN-VTA voxels at P<0.05, robust linear regression; P_{corrected}=0.038, permutation test; FIG. 7). In contrast, there was no significant effect for Digit Symbol score (194 of 1,807 SN-VTA voxels at P<0.05; P_{corrected}=0.121, permutation test) or HRSD (19 of 1,807 SN-VTA voxels at P<0.05; P_{corrected}=0.731, permutation test). A topographical analysis of the relationship between gait speed and NM-MRI CNR showed stronger relationships tended to occur in more medial (β_{|x|}=0.02, t₁₈₀₃=2.40, P=0.016), anterior (β_{y}=0.14, t₁₈₀₃=25.8, P=10⁻¹²⁴), and dorsal (β_{z}=−0.05, t₁₈₀₃=−6.62, P=10⁻¹⁰) SN-VTA voxels [multiple linear regression analysis predicting t statistic of gait speed effect across SN-VTA voxels as a function of their coordinates in x (absolute distance from the midline), y, and z directions: omnibus F_{3,1803}=297, P=10⁻¹⁵⁵].

Secondary Analyses Fail to Show Associations Between Baseline NM-MRI and Changes in Psychomotor Speed with L-DOPA Treatment

In a secondary analysis, the relationship between baseline NM-MRI signal and changes in psychomotor speed after 3 weeks of L-DOPA treatment in 15 patients with both baseline and post-treatment psychomotor evaluations (Study 2) was investigated. As a more stringent and spatially constrained test of this relationship, it was first determined if there was a relationship between changes in gait speed after 3 weeks of L-DOPA treatment and the average NM-MRI CNR in the 346 SN-VTA voxels that correlated positively with baseline gait speed (green voxels in FIG. 1). Here, were found no relationship between baseline NM-MRI CNR and the change in gait speed (t_{1,9}=0.71, P=0.49; robust linear regression testing for the effect of change in gait speed adjusting for baseline gait speed, age, gender, and education; FIG. 7). As a more lenient test of the hypothesis, a voxelwise analysis was performed in which, for each subject, the relationship was investigated between changes in gait speed and Digit Symbol scores after L-DOPA treatment with baseline NM-MRI CNR within the SN-VTA mask at each voxel (model 2). Again, no relationship was found between baseline NM-MRI CNR and the change in gait speed (64 of 1,807 SN-VTA voxels at P<0.05, robust linear regression testing for the effects of change in gait speed, change in Digit Symbol score, and change in HRSD adjusting for baseline gait speed, baseline Digit Symbol score, baseline HRSD age, gender, and education; Pcorrected=0.377, permutation test), change in Digit Symbol score (69 of 1,807 SN-VTA voxels at P<0.05, Pcorrected=0.361, permutation test), or change in HRSD (67 of 1,807 SN-VTA voxels at P<0.05, Pcorrected=0.371, permutation test).

Increases in NM-MRI CNR in the SN-VTA with L-DOPA Treatment

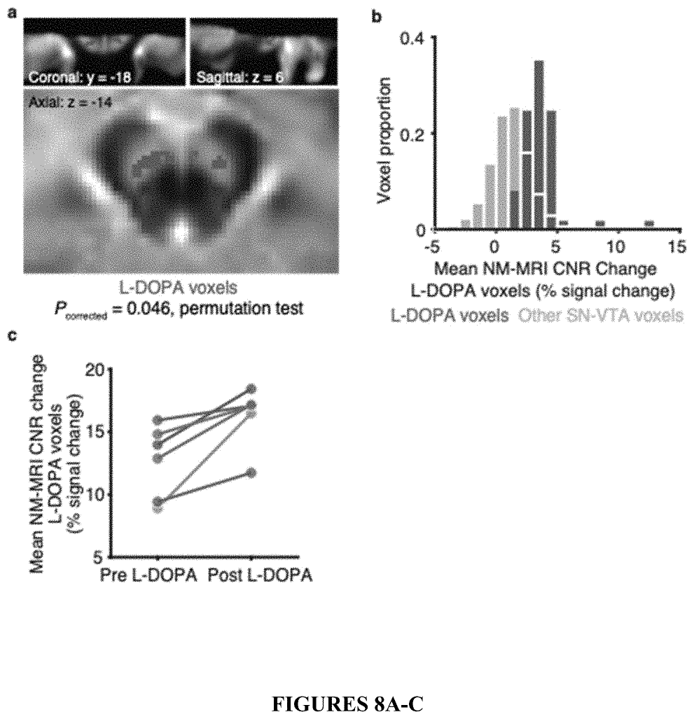

In an exploratory analysis, it was also investigated whether the NM-MRI signal changed after 3 weeks of L-DOPA treatment in the 6 patients with available baseline and post-treatment MRI data (Study 2 subset). To this end, a non-parametric voxelwise analysis was performed in which, for each subject, the difference in NM-MRI CNR at baseline and post-treatment within the SN-VTA mask at each voxel was tested. This revealed a set of SN-VTA voxels where NM-MRI CNR was significantly higher in the post-treatment scans (200 of 1,807 SN-VTA voxels at P<0.05, sign-rank test testing for the difference in NM-MRI CNR at baseline and post-treatment; Pcorrected=0.046, permutation test; FIG. 8).

Discussion

In this study, the relationship was investigated between NM-MRI data and psychomotor speed in older adults with LLD and found that lower NM-MRI signal in medial, anterior and dorsal parts of the SN-VTA complex was associated with slower gait speed. In a secondary analysis of a smaller sample of subjects who underwent L-DOPA treatment, it was not found that baseline NM-MRI predicted changes in psychomotor speed after treatment. Furthermore, in an exploratory analysis, it was observed that 3-week L-DOPA treatment was associated with significant increases in NM-MRI signal.

The finding of lower dopamine function, as indexed by lower NM-MRI signal, being associated with slower gait speed is consistent with the a priori hypotheses based on previous literature (19). For example, recent studies have identified a relationship between a genetic polymorphism of Catechol-O-methyltransferase (COMT, rs4680; which regulates tonic dopamine) and gait speed (30, 31). Additionally, in older patients with cerebral small vessel disease, gait decline has been attributed to reductions in nigrostriatal dopamine (32). More generally, a strong theoretical foundation implicating dopamine function of the dorsal basal ganglia in age-related motor dysfunction has been proposed (33), and supports the need for dopaminergic biomarkers in this area.

The finding that dopamine function indexed by NM-MRI signal was not associated with Digit Symbol scores was not consistent with the hypothesis or previous reports linking dopamine function and processing speed. The limited sample size (N=33) restricts the ability to conclude that there is no association between Digit Symbol scores and dopamine function, and studies in larger samples are required to address this. Dopamine is theoretically linked to processing speed (34), but empirical evidence correlating neuroimaging-based measures of dopamine signaling with performance on processing speed tasks is mixed. The largest study to date (N=181 healthy adults) showed no significant correlation between striatal raclopride PET D2-receptor binding and processing speed (35); although smaller studies have observed small, but significant, associations between processing speed and dopamine function (16, 36). The Applicant is not aware of any studies to have demonstrated significant correlations between dopamine signaling and Digit Symbol scores. Thus, while the Digit Symbol test's motor requirements and speed dependence is theoretically suggestive of a link to dopamine function, there may be more complexity involved (37). Furthermore, although motor speed and attention are impaired in both aging (38, 39) and depressed (40-42) populations, these deficits are often subtle and not detected through the Digit Symbol test (43); and the mechanisms for their impairment in these clinical populations may not be dopaminergic.

In secondary analyses of the smaller sample of subjects who underwent L-DOPA treatment (N=15), an association between baseline NM-MRI and changes in psychomotor speed after treatment were not found. This was in contrast with the hypothesis and could be due to a lack of statistical power from the small sample size. If these results hold in a larger sample size, it may suggest that baseline dopamine function is not predictive of L-DOPA efficacy regarding changes in psychomotor function.

In an exploratory analysis, a significant increase in NM-MRI signal after L-DOPA treatment was observed, supporting the notion that the L-DOPA treatment is likely increasing available striatal dopamine, but that participants are responding differently to that increase (11). It is unlikely that the observed changes are due to natural NM accumulation over time, because this age-related process occurs very slowly and should only be detectable over a substantially longer timescale than the 3-week period evaluated here (44). Furthermore, although the sample size is limited (N=6), the excellent reproducibility of NM-MRI suggests that any observed increase in NM-MRI signal is indeed due to an increase in NM concentration (27). This result provides further evidence supporting that NM-MRI measures dopamine function, including synthesis induced by L-DOPA (45). This result also suggests that NM-MRI may be surprisingly sensitive to changes in NM at shorter timescales than previously thought (46). Although caution is warranted given the limitations of the sample size and further investigation is needed, if replicated in a large sample, this finding suggests that NM-MRI could be well suited for monitoring of dopaminergic treatment response.

The results of the topographical analysis of the relationship between gait speed and NM-MRI signal showed that stronger relationships occurred in the medial, anterior, and dorsal areas of the SN-VTA. In contrast, NM-MRI data have shown that larger signal decreases in PD tend to predominate in more lateral, posterior and ventral voxels (20, 47). Furthermore, histopathological studies have also found that PD-related neuron loss occurs mainly in the ventrolateral tier of the SN (48, 49), with recent free water imaging studies identifying similar spatial patterns (50, 51). A recent study used NM-MRI to analyze the signal intensity of the SN in two motor subtypes of PD, with patients classified as either postural instability, gait difficulty dominant or tremor dominant, along with controls. Significant signal attenuation was detected in the lateral part of SN in both PD subtypes when compared with the controls, and severe signal attenuation was also observed in the medial part of SN in postural instability, gait difficulty dominant patients in comparison with the tremor dominant group (52). Taken together, the topological findings, in addition to the fact that slowed, depressed subjects typically do not manifest the clinical stigmata of PD (e.g., cog wheeling, freezing, tremor etc.), support that the sample of LLD patients is not likely a sample of subclinical PD patients.

Here, NM-MRI was used as a proxy marker for dopamine function and LDD-related alterations. This was supported by previous work showing that NM-MRI captures NM concentration in ex vivo tissue samples and that it correlates with increased dopamine transmission (20), consistent with the finding that enhancing dopamine synthesis results in increased NM accumulation (53, 54). Although a role of NM itself in the pathophysiology of LDD was not hypothesized, an involvement in Parkinson's disease has been proposed. NM is the main iron storage molecule in dopaminergic neurons of the SN and provides a neuroprotective effect by preventing the accumulation of cytosolic dopamine (53, 55). In conditions of iron overload, NM however can play a neurotoxic role (56) and NM released into the extracellular space can cause microglial activation and subsequent neurodegeneration (57). Given this, and while the results are interpreted to reflect changes in dopamine function associated with slowing and L-DOPA versus alterations in NM synthesis pathways per se, the latter possibility cannot be ruled out and should be examined in future work (e.g., combining PET dopamine and NM-MRI measures concurrently).

Some limitations of the current study are worth discussing. The open-label administration of L-DOPA in this study may have led to expectancy-based placebo effects, though some evidence suggests that these effects are diminished in older adults with depression relative to younger adults (58). Still, a portion of the improvements observed may be attributable to these expectations, as well as to therapeutic interactions with the research staff, or to spontaneous improvement. It is plausible that NM-MRI were not found to be predictive of treatment response because of these effects in combination with the relatively small sample size for this secondary analysis (N=15).

In conclusion, in patients with LLD, an association was found between NM-MRI signal in the SN-VTA and baseline gait speed, but not with changes in gait speed or processing speed after 3 weeks of L-DOPA treatment. Future work using a double-blind, placebo-controlled design with a larger sample is warranted to fully examine treatment effects with adequate power, determine the relationship between NM-MRI and placebo effects, and establish the time-course of NM-MRI signal changes under L-DOPA treatment.

References for Example 2

1. Friedhoff A J, Ballenger J, Bellack A S, Carpenter W T, Jr, Chui H C, Dobrof R, et al. Diagnosis and Treatment of Depression in Late Life. JAMA. 1992;268(8):1018-24.

2. Rothschild A J. The diagnosis and treatment of late-life depression. The Journal of clinical psychiatry. 1996;57:5-11.

3. Alexopoulos G S, Meyers B S, Young R C, Kakuma T, Feder M, Einhorn A, et al. Recovery in geriatric depression. Archives of General Psychiatry. 1996;53(4):305-12.

4. Sneed J R, Rutherford B R, Rindskopf D, Lane D T, Sackeim H A, Roose SP. Design makes a difference: a meta-analysis of antidepressant response rates in placebo-controlled versus comparator trials in late-life depression. The American Journal of Geriatric Psychiatry. 2008;16(1):65-73.

5. Sheline Y I, Barch D M, Garcia K, Gersing K, Pieper C, Welsh-Bohmer K, et al. Cognitive function in late life depression: relationships to depression severity, cerebrovascular risk factors and processing speed. Biological psychiatry. 2006;60(1):58-65.

6. Brown P J, Roose S P, Zhang J, Wall M, Rutherford B R, Ayonayon H N, et al. Inflammation, depression, and slow gait: a high mortality phenotype in later life. Journals of Gerontology Series A: Biomedical Sciences and Medical Sciences. 2016;71(2):221-7.

7. Rutherford B R, Taylor W D, Brown P J, Sneed J R, Roose SP. Biological aging and the future of geriatric psychiatry. Journals of Gerontology Series A: Biomedical Sciences and Medical Sciences. 2017;72(3):343-52.

8. Pimontel M A, Culang-Reinlieb M E, Morimoto S S, Sneed J R. Executive dysfunction and treatment response in late-life depression. International journal of geriatric psychiatry. 2012;27(9):893-9.

9. Kerse N, Flicker L, Pfaff J J, Draper B, Lautenschlager N T, Sim M, et al. Falls, depression and antidepressants in later life: a large primary care appraisal. PLoS One. 2008;3(6).

10. Wolinsky F D, Callahan C M, Fitzgerald J F, Johnson R J. Changes in functional status and the risks of subsequent nursing home placement and death. Journal of gerontology. 1993;48(3):594-101.

11. Rutherford B R, Slifstein M, Chen C, Abi-Dargham A, Brown P J, Wall M W, et al. Effects of L-DOPA Monotherapy on Psychomotor Speed and [11C] Raclopride Binding in High-Risk Older Adults With Depression. Biological psychiatry. 2019;86(3):221-9.

12. Rapp M A, Reischies F M. Attention and executive control predict Alzheimer disease in late life: results from the Berlin Aging Study (BASE). The American Journal of Geriatric Psychiatry. 2005;13(2):134-41.

13. Verghese J, Holtzer R, Lipton R B, Wang C. Quantitative gait markers and incident fall risk in older adults. The Journals of Gerontology: Series A. 2009;64(8):896-901.

14. Guralnik J M, Ferrucci L, Pieper C F, Leveille S G, Markides K S, Ostir G V, et al. Lower extremity function and subsequent disability: consistency across studies, predictive models, and value of gait speed alone compared with the short physical performance battery. The Journals of Gerontology Series A: Biological Sciences and Medical Sciences. 2000;55(4):M221-M31.

15. äckman L, Nyberg L, Lindenberger U, Li S-C, Farde L. The correlative triad among aging, dopamine, and cognition: current status and future prospects. Neuroscience & Biobehavioral Reviews. 2006;30(6):791-807.

16. Volkow N D, Gur RC, Wang G-J, Fowler J S, Moberg P J, Ding Y-S, et al. Association between decline in brain dopamine activity with age and cognitive and motor impairment in healthy individuals. Am J Psychiat. 1998;155(3):344-9.

17. Kaasinen V, Vilkman H, Hietala J, Nagren K, Helenius H, Olsson H, et al. Age-related dopamine D2/D3 receptor loss in extrastriatal regions of the human brain. Neurobiology of aging. 2000;21(5):683-8.

18. Eckart C, Bunzeck N. Dopamine modulates processing speed in the human mesolimbic system. Neuroimage. 2013;66:293-300.

19. Cham R, Studenski S, Perera S, Bohnen N. Striatal dopaminergic denervation and gait in healthy adults. Experimental brain research. 2008;185(3):391-8.

20. Cassidy C M, Zucca F A, Girgis R R, Baker S C, Weinstein J J, Sharp M E, et al. Neuromelanin-sensitive MRI as a noninvasive proxy measure of dopamine function in the human brain. Proceedings of the National Academy of Sciences. 2019;116(11):5108-17.

21. Chen X, Huddleston D E, Langley J, Ahn S, Barnum C J, Factor S A, et al. Simultaneous imaging of locus coeruleus and substantia nigra with a quantitative neuromelanin MRI approach. Magnetic resonance imaging. 2014;32(10):1301-6.

22. Zucca F A, Basso E, Cupaioli F A, Ferrari E, Sulzer D, Casella L, et al. Neuromelanin of the human substantia nigra: an update. Neurotoxicity research. 2014;25(1):13-23.

23. Zecca L, Shima T, Stroppolo A, Goj C, Battiston G, Gerbasi R, et al. Interaction of neuromelanin and iron in substantia nigra and other areas of human brain. Neuroscience. 1996;73(2):407-15.

24. Zecca L, Bellei C, Costi P, Albertini A, Monzani E, Casella L, et al. New melanic pigments in the human brain that accumulate in aging and block environmental toxic metals. Proceedings of the National Academy of Sciences. 2008;105(45):17567-72.

25. Sulzer D, Zecca L. Intraneuronal dopamine-quinone synthesis: a review. Neurotoxicity research. 1999;1(3):181-95.

26. Wechsler D. The Wechsler Memory Scale, San Antonio, Tex., Psychological Corp. Harcourt; 1997.

27. Wengler K, He X, Abi-Dargham A, Horga G. Reproducibility assessment of neuromelanin-sensitive magnetic resonance imaging protocols for region-of-interest and voxelwise analyses. Neurolmage. 2020;208:116457.

28. Slinker B K, Glantz S A. Multiple linear regression: accounting for multiple simultaneous determinants of a continuous dependent variable. Circulation. 2008;117(13):1732-7.

29. Eklund A, Nichols T E, Knutsson H. Cluster failure: Why fMRI inferences for spatial extent have inflated false-positive rates. Proceedings of the national academy of sciences. 2016;113(28):7900-5.

30. Hupfeld K E, Vaillancourt D E, Seidler R D. Genetic markers of dopaminergic transmission predict performance for older males but not females. Neurobiology of aging. 2018;66:180. e11-e21.

31. Rosano C, Metti A L, Rosso A L, Studenski S, Bohnen N I. Influence of Striatal Dopamine, Cerebral Small Vessel Disease, and Other Risk Factors on Age-Related Parkinsonian Motor Signs. The Journals of Gerontology: Series A. 2019;75(4):696-701.

32. Rosso A L, Bohnen N I, Launer L J, Aizenstein H J, Yaffe K, Rosano C. Vascular and dopaminergic contributors to mild parkinsonian signs in older adults. Neurology. 2018;90(3):e223-e9.

33. Clark B C, Woods A J, Clark L A, Criss C R, Shadmehr R, Grooms D R. The Aging Brain & the Dorsal Basal Ganglia: Implications for Age-Related Limitations of Mobility. Advances in Geriatric Medicine and Research. 2019;1(2):e190008.

34. Salthouse T A. Aging and measures of processing speed. Biological psychology. 2000;54(1-3):35-54.

35. Nyberg L, Karalija N, Salami A, Andersson M, Wållin A, Kaboovand N, et al. Dopamine D2 receptor availability is linked to hippocampal-caudate functional connectivity and episodic memory. Proceedings of the National Academy of Sciences. 2016;113(28):7918-23.

36. Vriend C, van Balkom T D, van Druningen C, Klein M, van der Werf Y D, Berendse H W, et al. Processing speed is related to striatal dopamine transporter availability in Parkinson's disease. Neurolmage: Clinical. 2020:102257.

37. Jaeger J. Digit symbol substitution test: the case for sensitivity over specificity in neuropsychological testing. Journal of clinical psychopharmacology. 2018;38(5):513.

38. Seidler R D, Bernard J A, Burutolu T B, Fling B W, Gordon M T, Gwin J T, et al. Motor control and aging: links to age-related brain structural, functional, and biochemical effects. Neuroscience & Biobehavioral Reviews. 2010;34(5):721-33.

39. Corti E J, Johnson A R, Riddle H, Gasson N, Kane R, Loftus A M. The relationship between executive function and fine motor control in young and older adults. Human movement science. 2017;51:41-50.

40. LeMoult J, Gotlib I H. Depression: A cognitive perspective. Clinical Psychology Review. 2019;69:51-66.

41. Li C-T, Lin C-P, Chou K-H, Chen I-Y, Hsieh J-C, Wu C-L, et al. Structural and cognitive deficits in remitting and non-remitting recurrent depression: a voxel-based morphometric study. Neuroimage. 2010;50(1):347-56.

42. Yaroslaysky I, Allard E S, Sanchez-Lopez A. Can't look away: Attention control deficits predict rumination, depression symptoms and depressive affect in daily life. Journal of Affective Disorders. 2019;245:1061-9.

43. Shura R D, Rowland J A, Martindale S L, Brearly T W, Delahanty M B, Miskey H M. Evaluating the motor slowing hypothesis of depression. Psychiatry research. 2017;252:188-95.

44. Zecca L, Fariello R, Riederer P, Sulzer D, Gatti A, Tampellini D. The absolute concentration of nigral neuromelanin, assayed by a new sensitive method, increases throughout the life and is dramatically decreased in Parkinson's disease. FEBS letters. 2002;510(3):216-20.

45. Tison F, Mons N, Geffard M, Henry P. The metabolism of exogenous L-dopa in the brain: an immunohistochemical study of its conversion to dopamine in non-catecholaminergic cells of the rat brain. Journal of neural transmission-Parkinson's disease and dementia section. 1991;3(1):27-39.

46. Matsuura K, Maeda M, Tabei K-i, Umino M, Kajikawa H, Satoh M, et al. A longitudinal study of neuromelanin-sensitive magnetic resonance imaging in Parkinson's disease. Neuroscience letters. 2016;633:112-7.

47. Sulzer D, Cassidy C, Horga G, Kang U J, Fahn S, Casella L, et al. Neuromelanin detection by magnetic resonance imaging (MRI) and its promise as a biomarker for Parkinson's disease. NPJ Parkinson's disease. 2018;4(1):11.

48. Damier P, Hirsch E, Agid Y, Graybiel A. The substantia nigra of the human brain: II. Patterns of loss of dopamine-containing neurons in Parkinson's disease. Brain. 1999;122(8):1437-48.

49. Fearnley J M, Lees A J. Ageing and Parkinson's disease: substantia nigra regional selectivity. Brain. 1991;114(5):2283-301.

50. Ofori E, Pasternak O, Planetta P J, Burciu R, Snyder A, Febo M, et al. Increased free water in the substantia nigra of Parkinson's disease: a single-site and multi-site study. Neurobiology of aging. 2015;36(2):1097-104.

51. Planetta P J, Ofori E, Pasternak O, Burciu R G, Shukla P, DeSimone J C, et al. Free-water imaging in Parkinson's disease and atypical parkinsonism. Brain. 2016;139(2):495-508.

52. Xiang Y, Gong T, Wu J, Li J, Chen Y, Wang Y, et al. Subtypes evaluation of motor dysfunction in Parkinson's disease using neuromelanin-sensitive magnetic resonance imaging. Neuroscience letters. 2017;638:145-50.

53. Sulzer D, Bogulaysky J, Larsen K E, Behr G, Karatekin E, Kleinman M H, et al. Neuromelanin biosynthesis is driven by excess cytosolic catecholamines not accumulated by synaptic vesicles. Proceedings of the National Academy of Sciences. 2000;97(22):11869-74.

54. Cebrián C, Zucca F A, Mauri P, Steinbeck J A, Studer L, Scherzer CR, et al. MHC-I expression renders catecholaminergic neurons susceptible to T-cell-mediated degeneration. Nature communications. 2014;5:3633.

55. Zucca F A, Segura-Aguilar J, Ferrari E, Munoz P, Paris I, Sulzer D, et al. Interactions of iron, dopamine and neuromelanin pathways in brain aging and Parkinson's disease. Progress in neurobiology. 2017;155:96-119.

56. Zecca L, Casella L, Albertini A, Bellei C, Zucca F A, Engelen M, et al. Neuromelanin can protect against iron-mediated oxidative damage in system modeling iron overload of brain aging and Parkinson's disease. Journal of neurochemistry. 2008;106(4):1866-75.

57. Zecca L, Wilms H, Geick S, Claasen J-H, Brandenburg L-O, Holzknecht C, et al. Human neuromelanin induces neuroinflammation and neurodegeneration in the rat substantia nigra: implications for Parkinson's disease. Acta neuropathologica. 2008;116(1):47-55.

58. Rutherford B R, Wall M M, Brown P J, Choo T-H, Wager T D, Peterson B S, et al. Patient expectancy as a mediator of placebo effects in antidepressant clinical trials. Am J Psychiat. 2017;174(2):135-42.

1-131. (canceled)

132. An apparatus, comprising:
a memory; and
a processor operatively coupled to the memory, the processor configured to:
receive imaging information of a subject, the imaging information including Neuromelanin (NM)-Magnetic Resonance Imaging (NM-MRI) scans of a dopamine-associated brain region of interest of the subject;
receiving control scans of the dopamine-associated brain region of interest of a control subject;
determine a first NM concentration using a voxelwise analysis based on the NM-MRI scans, the first NM concentration in the dopamine-associated brain region of interest of the subject;
determine a second NM concentration using a voxelwise analysis based on the control scans, the second NM concentration in the dopamine-associated brain region of interest of the control subject; and
determine, based on the first NM concentration and the second NM concentration, an increase or a decrease of dopamine function associated with the subject.

133. The apparatus of claim 132, wherein the processor is configured to determine an increase in dopamine function if the first NM concentration is greater than the second NM concentration, and the processor is configured to determine a decrease in dopamine function if the first NM concentration is lesser than the second NM concentration.

134. The apparatus of claim 132, wherein the voxelwise analysis includes determining at least one topographical pattern within the brain region of interest, the at least one topological pattern being based on a change in cell number in the brain region of interest.

135. The apparatus of claim 132, wherein the NM-MRI scans are associated with the subject's performance on a cognitive task.

136. The apparatus of claim 132, wherein the brain region of interest is at least one of (1) substantia nigra (SN), ventral SN, lateral SN, ventrolateral SN, substantia nigra pars compacta (SNpc), substantia nigra pars reticulata (SNpr), or ventral tegmental area (VTA).

137. The apparatus of claim 132, wherein the subject has or is suspected of having a dopamine function-related disorder, or at least one of a schizophrenia spectrum disorder, psychotic illness, addiction disorder, depression, late-life depression, bipolar disorder, Huntington's disease, Parkinson's disease, a movement disorder, a psychomotor slowing, a neuropsychiatric disorder, or a cocaine use disorder.

138. The apparatus of claim 135, wherein the cognitive task is configured to assess catecholamine-related processes.

139. The apparatus of claim 138, wherein the catecholamine-related processes include at least one of dopamine-related processes or reward related processes.

140. The apparatus of claim 132, wherein the NM-MRI scans are based on at least one or functional MRI, structural MRI, or blood oxygen level dependent (BOLD) functional MRI.

141. A non-transitory processor-readable medium storing code representing instructions to be executed by a processor, the instructions comprising code to cause the processor to:
receive imaging information of a brain region of interest of a subject, the imaging information including Neuromelanin (NM)-Magnetic Resonance Imaging (NM-MRI) scans of the brain region of interest of the subject;
determine a NM signal associated with the brain region of interest of the subject using voxelwise analysis based on the NM-MRI scans;
compare the NM signal with a control signal obtained from control scans known to be not associated with a neuropsychiatric disorder; and
determine if the subject has developed or is at risk of developing the neuropsychiatric disorder based on the comparison between the NM signal and the control signal.

142. The non-transitory processor-readable medium of claim 141, wherein the instructions further comprise code to cause the processor to:
determine, if the NM signal is different beyond a predefined threshold compared to the control signal, that the subject has or is at risk of developing the neuropsychiatric disorder, and
determining, if the NM signal is similar, within a predefined threshold, compared to the control signal obtained from the control scans, that the subject does not have or is not at risk of developing the neuropsychiatric disorder.

143. The non-transitory processor-readable medium of claim 141, wherein the voxelwise analysis includes determining at least one topographical pattern within the brain region of interest.

144. The non-transitory processor-readable medium of claim 143, wherein the at least one topological pattern is based on a change in cell number in the brain region of interest.

145. The non-transitory processor-readable medium of claim 141, wherein the NM-MRI scans are associated with the subject's performance on a cognitive task.

146. The non-transitory processor-readable medium of claim 141, wherein the brain region of interest is at least one of (1) substantia nigra (SN), ventral SN, lateral SN, ventrolateral SN, substantia nigra pars compacta (SNpc), substantia nigra pars reticulata (SNpr), or ventral tegmental area (VTA).

147. A method for assessing development of a cognitive disorder, the method comprising:
receiving imaging information including Neuromelanin (NM)-Magnetic Resonance Imaging (NM-MRI) scans of a subject's dopamine-associated brain region of interest;
determining a NM signal from the subject's dopamine-associated brain region of interest using a voxelwise analysis based on the NM-MRI scans;
determining, if the NM signal is different beyond a predefined threshold compared to a control signal obtained from control scans known to be not associated with a cognitive disorder, that the subject has or is at risk of developing a cognitive disorder, and
determining, if the NM signal is similar, within a predefined threshold, compared to the control signal obtained from the control scans, that the subject does not have or is not at risk of developing the cognitive disorder.

148. The method of claim 147, wherein the NM-MRI scans are associated with the subject's performance on a cognitive task.

149. The method of claim 148, wherein the cognitive task is configured to assess catecholamine-related processes.

150. The method of claim 147, wherein the catecholamine-related processes include at least one of dopamine-related processes or reward related processes.

151. The method of claim 147, wherein the brain region of interest is at least one of (1) substantia nigra (SN), ventral SN, lateral SN, ventrolateral SN, substantia nigra pars compacta (SNpc), substantia nigra pars reticulata (SNpr), or ventral tegmental area (VTA).
